SEQUENCE LISTING (2 sequences)

SEQ ID NO: 1 - protein, 10 aa, Artificial Sequence
  Chemically Synthesized
SFNSYELGSL

SEQ ID NO: 2 - protein, 11 aa, Artificial Sequence
  Chemically Synthesized
YGRKKRRQRRR

(19) United States
(12) Patent Application Publication
(10) Pub. No.: US 2020/0113979 A1
(43) Pub. Date: Apr. 16, 2020

(54) Protein Kinase C-delta targeted therapy for treating radiation injury

(71) Applicant: Temple University-Of The Commonwealth System of Higher Education, Philadelphia, PA (US)

(72) Inventors: Mohammad F. Kiani, Wynnewood, PA (US); Laurie E. Kilpatrick, Narberth, PA (US); Yuan Tang, Blue Bell, PA (US); Fariborz Soroush, Drexel Hill, PA (US)

(21) Appl. No.: 16/621,720

(22) PCT Filed: Jun. 12, 2018
(86) PCT No.: PCT/US18/36987

§ 371 Date: Dec. 12, 2019

Related U.S. Application Data
(60) Provisional application No. 62/518,058, filed on Jun. 12, 2017.

(51) Int. Cl. A61K 38/45; A61K 38/16; A61P 43/00

(52) U.S. Cl. CPC A61K 38/45; C12Y 207/10002; A61P 43/00; A61K 38/162

(57) ABSTRACT

The present invention provides compositions and methods for enhancing the radiation resistance of a cell by modulating PKCδ and methods of preventing or treating radiation-mediated injury.

### CROSS REFERENCE TO RELATED APPLICATIONS

This application claims priority to U.S. Provisional Application No. 62/518,058, filed Jun. 12, 2017 which is hereby incorporated by reference herein in its entirety.

### BACKGROUND OF THE INVENTION

In the event of a radiological catastrophe, affected individuals will be exposed to a wide range of radiation doses with the extent of injury depending on the victims' distance from the epicenter, the duration of exposure, and their inherent sensitivity/resistance to ionizing radiation (IR). Radiation induced damage to the vascular endothelium plays a key role in the early and late onset of radiation induced pathologies in several organs (Wang et al., 2007, World journal of gastroenterology, 13:3047-3055) and is an active participant in the recruitment and activation of neutrophils through the production of chemokines/cytokines and expression of adhesion molecules (Molla et al., 1999, Int. J. Radiat. Oncol. Biol. Phys, 45:1011-1018; Wood et al., 2005, Neurosurgery, 57:1282-1288; Kiani et al., 2002, Pharm. Res, 19:1317-1322; Yuan et al., 2005, Radiat. Res, 163:544-551). Downregulating endothelial inflammatory activation protects normal tissue from radiation-induced damage (Korpela and Liu, 2014, Radiat Oncol, 9:266; Williams and McBride, 2011, Int J Radiat Biol, 87:851-868).

Key to radiation-induced tissue damage is the excessive migration of activated neutrophils across the vascular endothelium, (Flanders et al., 2008, Am J Pathol, 173:68-76; Francois et al., 2013, Biomed Res Int, 2013:123241) and reduction in neutrophil infiltration is associated with better outcomes following skin irradiation (Korpela and Liu, 2014, Radiat Oncol, 9:266). Post radiation injury, systemic inflammation leads to increased endothelial cell-adhesion molecule expression resulting in increased neutrophil-endothelial cell interaction, vascular endothelial cell damage, and organ dysfunction (Molla et al., 1999, Int. J. Radiat. Oncol. Biol. Phys, 45:1011-1018; Wood et al., 2005, Neurosurgery, 57:1282-1288; Kiani et al., 2002, Pharm. Res, 19:1317-1322; Yuan et al., 2005, Radiat. Res, 163:544-551; Prabhakarpandian et al., 2001, Microcirc, 8:355-364; Panes et al., 1995, Gastroenterology, 108:1761-1769; Ansari et al., 2007, Radiat. Res, 167:80-86). Neutrophil-endothelial cell interaction starts with neutrophil rolling on the endothelium which is mediated by selectins, whereas firm adhesion and migration into the tissue are mediated by a combination of integrins/immunoglobulins and chemoattractants in the tissue. Due to the significance of the neutrophil-endothelial cell interactions, and given the complexity of existing in vivo models of the inflammatory process, several in vitro models have been developed to study different aspects of the neutrophil adhesion cascade. Unfortunately, for the most part these models cannot characterize adhesion and migration in a single assay. To overcome these limitations, a novel biomimetic microfluidic assay (bMFA) has been developed that resolves and facilitates real-time assessment of individual steps including rolling, firm arrest, spreading and migration of neutrophils into the extra-vascular tissue space in a single system which allows direct observation and quantification of neutrophil-endothelial cell interaction over time in a realistic microvasculature geometry with physiological shear conditions (Prabhakarpandian et al., 2011, Microvasc Res, 82:210-220; Prabhakarpandian et al., 2008, Biomed Microdevices, 10:585-595; Tousi et al., 2010, MicrovascRes, 80(3):384-388). This is the first in vitro system which realistically models in vivo geometrical features (e.g., bifurcations, vascular morphology) and flow conditions (e.g., converging or diverging flows at bifurcations) of the microvasculature and allows for interrogation of the role of various factors in IR-induced leukocyte-endothelial cell interaction and endothelial cell damage (Rosano et al., 2009, Biomed Microdevices, 11:1051-1057). Thus, an integrated microfluidic assay was utilized to study specific steps in the leukocyte adhesion cascade induced by exposure to radiation.

While the use of Neupogen and Neulasta for treating hematopoietic acute radiation syndrome (H-ARS) was recently approved by the FDA, treatment strategies for radiation-induced vascular injury are largely supportive and there are no specific pharmacologic therapies available that protect from radiation-mediated tissue damage (Satyamitra et al., 2016, Radiation research, 186:99-111; Singh and Seed, 2017, Int J Radiat Biol, 1-19). There is thus a need in the art for novel therapies for treating radiation injury. The present invention addresses this unmet need in the art.

### SUMMARY OF THE INVENTION

In one embodiment, the invention relates to a method of treating or preventing radiation-induced injury comprising administering a PKCδ inhibitor to a subject in need thereof.

In one embodiment, the PKCδ inhibitor is a PKCδ-TAT peptide inhibitor.

In one embodiment, the PKCδ inhibitor is administered to a subject undergoing a radiation-based therapy.

In one embodiment, the PKCδ inhibitor is administered to a subject exposed to high levels of radiation.

In one embodiment, the subject is human.

In one embodiment, the invention relates to a method of decreasing neutrophil migration in a subject in need thereof, comprising administering a PKCδ inhibitor to the subject.

In one embodiment, the PKCδ inhibitor is a PKCδ-TAT peptide inhibitor.

In one embodiment, the PKCδ inhibitor is administered to a subject undergoing a radiation-based therapy.

In one embodiment, the PKCδ inhibitor is administered to a subject exposed to high levels of radiation.

In one embodiment, the subject is human.

In one embodiment, the invention relates to the use of a PKCδ inhibitor in the manufacture of a medicament for the treatment or prevention of radiation-induced injury.

In one embodiment, the PKCδ inhibitor is a PKCδ-TAT peptide inhibitor.

In one embodiment, the the radiation-induced injury is associated with a radiation-based therapy.

In one embodiment, the the radiation-induced injury is associated with exposure to high levels of radiation.

### BRIEF DESCRIPTION OF THE DRAWINGS

The following detailed description of exemplary embodiments of the invention will be better understood when read in conjunction with the appended drawings. It should be understood, however, that the invention is not limited to the precise arrangements and instrumentalities of the exemplary embodiments shown in the drawings.

FIG. 1, comprising FIG. 1A through FIG. 1E, depicts images of various stages of the experimental design. FIG. 1A depicts an exemplary intravital microscopy image used to map microvascular networks in animals. These maps are then used to fabricate the vascular network on PDMS and assemble the biomimetic microfluidic assay (bMFA). FIG. 1B depicts an image of the bMFA. FIG. 1C depicts a drawing showing that the bMFA includes vascular channels that are connected to the tissue compartment through a 3 μm barrier.

FIG. 2 depicts a diagram showing the timeline of experiments. bMFA is prepared and seeded with HUVEC. After endothelial cells reach confluency under shear flow and form a complete lumen in bMFA (usually in 48 hours after seeding) they are treated with ionizing radiation. At 24 hours post-IR, the cells are treated with either PKCδ-TAT peptide inhibitor (PKCδ-i) or vehicle. At 48 hours post-IR, fMLP is added to the tissue compartment and interaction of either labeled neutrophils or fluorescent microparticles with endothelial cells are studied.

FIG. 3 depicts exemplary experimental results demonstrating that radiation exposure induces PKCδ activation through phosphorylation and translocation. HUVEC cells were exposed to varying levels of radiation and PKCδ Ser643 phosphorylation and translocation to membrane fractions were determined as described in Methods and Materials. A) Representative western blot of PKCδ membrane translocation response to 0, 0.5, 2 and 5 Gy and VE-cadherin as a marker for membrane fractions (n=4 separate experiments). Membrane extracts were prepared as described in Methods and Materials. B) Densitometry analysis of PKCδ (Ser643) in the membrane fraction. Values are expressed in arbitrary densitometry units (ADU); Mean±SEM (n=4); One-way ANOVA *P<0.05, **P<0.01

FIG. 4, comprising FIG. 4A through FIG. 4C, depicts images of endothelial cells with various treatments. FIG. 4A depicts an image demonstrating that endothelial cells are aligned in the direction of flow under control conditions. FIG. 4B depicts an image demonstrating that in response to 5Gy IR, endothelial cells are not as well aligned and denuded (solid arrows). FIG. 4C depicts an image demonstrating that inhibition of PKCδ 24 hours post-IR prevents denuding of endothelial cells which align in the direction of flow (open arrow). Cells are stained with VE-cadherin (adherens junction); phalloidin (actin filament); and Hoechst 33342 (cell nucleus).

FIG. 5 depicts exemplary experimental results demonstrating that dextran permeability of endothelial cells exposed to irradiation is significantly increased. Treatment of cells with PKCδ-TAT inhibitor (PKCδ-i) restores permeability to control levels (0 Gy). Data are normalized with respect to the permeability of HUVEC with no treatment. Mean±SEM (n=3), two-way ANOVA, *p<0.05, **p<0.01.

FIG. 6 depicts exemplary experimental results demonstrating that neutrophil migration across irradiated endothelial cells increases over time by up to 20 fold at 60 minutes. PKCδ inhibition with PKCδ-TAT inhibitor (PKCδ-i) at 24 hours post-IR significantly reduces neutrophil migration by up to 82% after 60 minutes. Mean±SEM (n=3), two-way ANOVA, *p<0.05, **p<0.01, ***p<0.001.

FIG. 7 depicts exemplary experimental results demonstrating that neutrophil adhesion to endothelial cells significantly increases post-IR, especially in vessels with lower shear rates and near bifurcations. Inhibition of PKCδ with PKCδ-TAT inhibitor (PKCδ-i) significantly reduces neutrophil adhesion at shear rates lower than 60 s⁻¹ and at bifurcations. Mean±SEM (n=3), two-way ANOVA, *p<0.05, **p<0.01, ***p<0.001.

FIG. 8 depicts exemplary experimental results demonstrating adhesion of mAb-coated microparticles to endothelial cells. Adhesion of anti-ICAM-1, anit-VCAM-1, and anti-P-selectin, but not anti-E-selectin, coated microparticles to endothelial cells significantly increases post-IR as compared to control. Inhibition of PKCδ with PKCδ-TAT inhibitor (PKCδ-i) 24 hours post-IR significantly reduces adhesion of anti-ICAM-1, anti-VCAM-1, and anti-P-selectin coated microparticles to endothelial cells. Mean±SEM (n=3), one-way ANOVA, *p<0.05, ***p<0.001.

FIG. 9, comprising FIG. 9A to FIG. 9B, depicts effects of systemic inflammation (sepsis) on lung PKCδ phosphorylation and nuclear translocation. FIG. 9A depicts an analysis of lung nuclear extracts 24 hours post sham, cecal ligation and puncture (CLP)+PBS or CLP+PKCδ-TAT inhibitor. Mean±SEM (N=7) in arbitrary densitometry units (ADU). *P<0.05 sham vs. CLP+PBS, **P<0.05 CLP+PBS vs CLP+PKCδTAT. HDAC2-nuclear marker. FIG. 9B depicts an analysis PKCδ Tyr 155 phosphorylation in lungs 24 hour post sham & CLP±PKCδ inhibitor. Cells are stained with PKCδ PhosphoTyr155, endothelium (RECA), and DAPI nuclear staining.

FIG. 10 depicts exemplary experimental results demonstrating ICAM-1 is upregulated in response to 5Gy IR in human dermal microvascular endothelial cells (HDMEC), human umbilical vein endothelial cells (HUVEC), and transformed human microvascular endothelial cells (HMEC-1). In contrast, E-selectin is only upregulated on irradiated HDMEC. (Mean±SEM, N=5, *p<0.05, **p<0.01)

FIG. 11 depicts the results of exemplary intravital microscopic studies in the cremaster muscle venules indicate that permeability to FITC-dextran 4.4-, 38.2-, 70, and 150-kDa increases significantly 24 hours after 20 Gy local irradiation. (*p<0.05, N=5).

FIG. 12 depicts exemplary experimental results demonstrating leukocyte-endothelial interaction on a chip. The biomimetic microfluidic assay (bMFA) consists of an endothelialized microvascular network in communication with a tissue compartment via a 3 μm porous barrier. As shown in the fluorescent images, the entire rolling, adhesion of migration of leukocytes can be directly observed in bMFA.

FIG. 13 depicts exemplary experimental results demonstrating neutrophil adhesion in bMFA is similar to leukocyte adhesion in vivo in mouse cremaster muscle. Distribution of the number of adhered leukocytes (in vivo, black bars) and neutrophils (in vitro, grey bars) as a function of distance from the nearest bifurcation. Both histograms are skewed to the left indicating preferential adhesion near bifurcations. (mean±SEM; N=3).

FIG. 14 depicts exemplary experimental results demonstrating transendothelial electrical resistance (TEER) levels in microvascular endothelial cells increased significantly under shear flow compared to static conditions (Mean±SEM, N=3, *p<0.05, **p<0.01).

FIG. 15 depicts exemplary experimental results demonstrating sepsis-induced lung injury is reduced in PKCδ KO and PKCδY155F KI mice compared to WT mice. H&E-stained lung sections obtained 24 hours post-sham and sepsis (CLP) surgery from WT-Sham, WTCLP, PKCδ KC KO-CLP and PKCδ Y155F KI CLP mice.

FIG. 16 depicts exemplary experimental results demonstrating sepsis-induced MPO levels are reduced in PKCδ KO and PKCδY155F KI mice. Lung MPO levels in WTsham and CLP (black), PKCδ KO (white) and PKCδ Y155F KI (gray) mice. Values are expressed as rfu/min/mg.

### DETAILED DESCRIPTION

The invention provides for compositions and methods for inhibiting Protein kinase C delta (PKCδ). The invention is based on the discovery that PKCδ serves to prevent tissue injury and offers a unique therapeutic target for the treatment of radiation injury. That is, the invention is based in part on the discovery that PKCδ inhibition dramatically attenuates radiation-induced leukocyte adhesion and migration as well as vascular endothelial permeability.

The present invention relates in part to enhancing the radiation resistance of a cell by modulating PKCδ and/or downstream targets and, consequently, attenuating radiation-mediated injury. Accordingly, in one embodiment, the invention provides compositions and methods for modulating PKCδ in cells. In one embodiment, the invention provides compositions and methods for modulating PKCδ in a subject experiencing or at risk of increased radiation exposure. In one embodiment, a subject experiencing or at risk of increased radiation exposure has experienced a catastrophic radiation event such as, but not limited to, a nuclear power plant failure. Other subjects that are at increased risk of radiation exposure include, but are not limited to, subjects undergoing radiation therapy for the treatment of cancer, subjects who frequent or are employed at altitudes above approximately 20,000 feet above sea level (e.g., pilots, flight attendants, astronauts), subjects who frequent or are employed underground (e.g, miners), and subjects who frequent or are employed at a facility that processes radioactive material (e.g, a uranium processing facility).

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

A “therapeutic” treatment is a treatment administered to a subject who exhibits signs of pathology, for the purpose of diminishing or eliminating those signs.

As used herein, “treating a disease or disorder” means reducing the frequency with which a symptom of the disease or disorder is experienced by a patient.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates in part to the use of Protein kinase C delta (PKCδ) inhibitors in a method of preventing or treating radiation-induced damage.

### Compositions

In one embodiment, the invention comprises a composition for enhancing the radiation resistance of a cell comprising an inhibitor of PKCδ. The inhibitor of PKCδ can be a pan-PKC inhibitor or a PKCδ-specific inhibitor. In one embodiment, the inhibitor of PKCδ is a pan-PKC inhibitor against one or more of PKCα, PKCβ, PKCγ and PKCδ. Exemplary PKC inhibitors that can be used according to the methods of the invention include, but are not limited to, Go 6983 and rottlerin.

In various embodiments, the present invention includes compositions and methods of treating or preventing radiation induced damage, or a disease or disorder associated therewith, in a subject. In various embodiments, the composition for treating cancer comprises an inhibitor of PKCδ. In one embodiment, the inhibitor of the invention decreases the amount of PKCδ polypeptide, the amount of PKCδ mRNA, the amount of PKCδ activity, or a combination thereof.

It will be understood by one skilled in the art, based upon the disclosure provided herein, that a decrease in the level of PKCδ encompasses the decrease in the expression, including transcription, translation, or both. The skilled artisan will also appreciate, once armed with the teachings of the present invention, that a decrease in the level of PKCδ includes a decrease in the activity of PKCδ. Thus, decrease in the level or activity of PKCδ includes, but is not limited to, decreasing the amount of polypeptide of PKCδ, and decreasing transcription, translation, or both, of a nucleic acid encoding PKCδ; and it also includes decreasing any activity of PKCδ as well.

In one embodiment, the invention provides a generic concept for inhibiting PKCδ as an anti-tumor therapy. In one embodiment, the composition of the invention comprises an inhibitor of PKCδ. In one embodiment, the inhibitor is selected from the group consisting of a small interfering RNA (siRNA), a microRNA, an antisense nucleic acid, a ribozyme, an expression vector encoding a transdominant negative mutant, an intracellular antibody, a peptide and a small molecule.

One skilled in the art will appreciate, based on the disclosure provided herein, that one way to decrease the mRNA and/or protein levels of PKCδ in a cell is by reducing or inhibiting expression of the nucleic acid encoding PKCδ. Thus, the protein level of PKCδ in a cell can also be decreased using a molecule or compound that inhibits or reduces gene expression such as, for example, siRNA, an antisense molecule or a ribozyme. However, the invention should not be limited to these examples.

In one embodiment, siRNA is used to decrease the level of PKCδ. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, Pa. (2003); and Gregory J. Hannon, Ed., RNAi A Guide to Gene Silencing, Cold Spring Harbor Laboratory Press, Cold Spring Harbor, N.Y. (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the present invention also includes methods of decreasing levels of PKCδ at the protein level using RNAi technology.

In other related aspects, the invention includes an isolated nucleic acid encoding an inhibitor, wherein an inhibitor such as an siRNA or antisense molecule, inhibits PKCδ, a derivative thereof, a regulator thereof, or a downstream effector, operably linked to a nucleic acid comprising a promoter/regulatory sequence such that the nucleic acid is preferably capable of directing expression of the protein encoded by the nucleic acid. Thus, the invention encompasses expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York) and as described elsewhere herein. In another aspect of the invention, PKCδ or a regulator thereof, can be inhibited by way of inactivating and/or sequestering one or more of PKCδ, or a regulator thereof. As such, inhibiting the effects of PKCδ can be accomplished by using a transdominant negative mutant.

In another aspect, the invention includes a vector comprising an siRNA or antisense polynucleotide. Preferably, the siRNA or antisense polynucleotide is capable of inhibiting the expression of PKCδ. The incorporation of a desired polynucleotide into a vector and the choice of vectors is well-known in the art as described in, for example, Sambrook et al., supra.

An siRNA polynucleotide is an RNA nucleic acid molecule that interferes with RNA activity that is generally considered to occur via a post-transcriptional gene silencing mechanism. An siRNA polynucleotide preferably comprises a double-stranded RNA (dsRNA) but is not intended to be so limited and may comprise a single-stranded RNA (see, e.g., Martinez et al., 2002 Cell 110:563-74). The siRNA polynucleotide included in the invention may comprise other naturally occurring, recombinant, or synthetic single-stranded or double-stranded polymers of nucleotides (ribonucleotides or deoxyribonucleotides or a combination of both) and/or nucleotide analogues as provided herein (e.g., an oligonucleotide or polynucleotide or the like, typically in 5′ to 3′ phosphodiester linkage). Accordingly it will be appreciated that certain exemplary sequences disclosed herein as DNA sequences capable of directing the transcription of the siRNA polynucleotides are also intended to describe the corresponding RNA sequences and their complements, given the well established principles of complementary nucleotide base-pairing.

Preferred siRNA polynucleotides comprise double-stranded polynucleotides of about 18-30 nucleotide base pairs, about 18, about 19, about 20, about 21, about 22, about 23, about 24, about 25, about 26, or about 27 base pairs. SiRNA polynucleotides may include one or more siRNA polynucleotide molecules that may differ (e.g., by nucleotide insertion or deletion) in length by one, two, three, four or more base pairs as a consequence of variability in processing, in biosynthesis, or in artificial synthesis of the siRNA. The siRNA polynucleotide of the present invention may also comprise a polynucleotide sequence that exhibits variability by differing (e.g., by nucleotide substitution, including transition or transversion) at one, two, three or four nucleotides from a particular sequence. These differences can occur at any of the nucleotide positions of a particular siRNA polynucleotide sequence, depending on the length of the molecule, whether situated in a sense or in an antisense strand of the double-stranded polynucleotide. The nucleotide difference may be found on one strand of a double-stranded polynucleotide, where the complementary nucleotide with which the substitute nucleotide would typically form hydrogen bond base pairing, may not necessarily be correspondingly substituted. In one embodiment, the siRNA polynucleotides are homogeneous with respect to a specific nucleotide sequence.

Based on the present disclosure, it should be appreciated that the siRNAs of the present invention may effect silencing of PKCδ expression to different degrees. The siRNAs thus must first be tested for their effectiveness. Selection of siRNAs are made therefrom based on the ability of a given siRNA to interfere with or modulate the expression of PKCδ. Accordingly, identification of specific siRNA polynucleotide sequences that are capable of interfering with expression of a desired target polypeptide requires production and testing of each siRNA. Methods for testing each siRNA and selection of suitable siRNAs for use in the present invention are known in the art.

One skilled in the art will appreciate, based on the disclosure provided herein, that one way to decrease the mRNA and/or protein level of PKCδ in a cell is by reducing or inhibiting expression of the nucleic acid encoding PKCδ, the protein level of PKCδ in a cell can also be decreased using a molecule or compound that inhibits or reduces gene expression such as, for example, an antisense molecule or a ribozyme.

In a preferred embodiment, the modulating sequence is an antisense nucleic acid sequence which is expressed by a plasmid vector. The antisense expressing vector is used to transfect a mammalian cell or the mammal itself, thereby causing reduced endogenous expression of a desired regulator in the cell. However, the invention should not be construed to be limited to inhibiting expression of PKCδ by transfection of cells with antisense molecules. Rather, the invention encompasses other methods known in the art for inhibiting expression or activity of a protein in the cell including, but not limited to, the use of a ribozyme, the expression of a non-functional regulator (i.e. transdominant negative mutant) and use of an intracellular antibody.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053). Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). A major advantage of this approach is the fact that ribozymes are sequence-specific.

In another aspect of the invention, the regulator can be inhibited by way of inactivating and/or sequestering the regulator. As such, inhibiting the effects of a regulator can be accomplished by using a transdominant negative mutant. Alternatively an antibody specific for the desired regulator, otherwise known as an antagonist to the regulator may be used. In one embodiment, the antagonist is a protein and/or compound having the desirable property of interacting with a binding partner of the regulator and thereby competing with the corresponding wild-type regulator. In another embodiment, the antagonist is a protein and/or compound having the desirable property of interacting with the regulator and thereby sequestering the regulator.

### Antibodies

As will be understood by one skilled in the art, any antibody that can recognize and bind to an antigen of interest is useful in the present invention. That is, the antibody can inhibit PKCδ to provide a beneficial effect.

Methods of making and using antibodies are well known in the art. For example, polyclonal antibodies useful in the present invention are generated by immunizing rabbits according to standard immunological techniques well-known in the art (see, e.g., Harlow et al., 1988, In: Antibodies, A Laboratory Manual, Cold Spring Harbor, N.Y.). Such techniques include immunizing an animal with a chimeric protein comprising a portion of another protein such as a maltose binding protein or glutathione (GSH) tag polypeptide portion, and/or a moiety such that the antigenic protein of interest is rendered immunogenic (e.g., an antigen of interest conjugated with keyhole limpet hemocyanin, KLH) and a portion comprising the respective antigenic protein amino acid residues. The chimeric proteins are produced by cloning the appropriate nucleic acids encoding the marker protein into a plasmid vector suitable for this purpose, such as but not limited to, pMAL-2 or pCMX.

However, the invention should not be construed as being limited solely to methods and compositions including these antibodies or to these portions of the antigens. Rather, the invention should be construed to include other antibodies, as that term is defined elsewhere herein, to antigens, or portions thereof. Further, the present invention should be construed to encompass antibodies, inter alia, bind to the specific antigens of interest, and they are able to bind the antigen present on Western blots, in solution in enzyme linked immunoassays, in fluorescence activated cells sorting (FACS) assays, in magenetic-actived cell sorting (MACS) assays, and in immunofluorescence microscopy of a cell transiently transfected with a nucleic acid encoding at least a portion of the antigenic protein, for example.

One skilled in the art would appreciate, based upon the disclosure provided herein, that the antibody can specifically bind with any portion of the antigen and the full-length protein can be used to generate antibodies specific therefor. However, the present invention is not limited to using the full-length protein as an immunogen. Rather, the present invention includes using an immunogenic portion of the protein to produce an antibody that specifically binds with a specific antigen. That is, the invention includes immunizing an animal using an immunogenic portion, or antigenic determinant, of the antigen.

Once armed with the sequence of a specific antigen of interest and the detailed analysis localizing the various conserved and non-conserved domains of the protein, the skilled artisan would understand, based upon the disclosure provided herein, how to obtain antibodies specific for the various portions of the antigen using methods well-known in the art or to be developed.

The skilled artisan would appreciate, based upon the disclosure provided herein, that that present invention includes use of a single antibody recognizing a single antigenic epitope but that the invention is not limited to use of a single antibody. Instead, the invention encompasses use of at least one antibody where the antibodies can be directed to the same or different antigenic protein epitopes.

The generation of polyclonal antibodies is accomplished by inoculating the desired animal with the antigen and isolating antibodies which specifically bind the antigen therefrom using standard antibody production methods such as those described in, for example, Harlow et al. (1988, In: Antibodies, A Laboratory Manual, Cold Spring Harbor, N.Y.).

Monoclonal antibodies directed against full length or peptide fragments of a protein or peptide may be prepared using any well known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art, and is described, for example, in Wright et al. (1992, Critical Rev. Immunol. 12:125-168), and the references cited therein. Further, the antibody of the invention may be “humanized” using the technology described in, for example, Wright et al., and in the references cited therein, and in Gu et al. (1997, Thrombosis and Hematocyst 77:755-759), and other methods of humanizing antibodies well-known in the art or to be developed.

The present invention also includes the use of humanized antibodies specifically reactive with epitopes of an antigen of interest. The humanized antibodies of the invention have a human framework and have one or more complementarity determining regions (CDRs) from an antibody, typically a mouse antibody, specifically reactive with an antigen of interest. When the antibody used in the invention is humanized, the antibody may be generated as described in Queen, et al. (U.S. Pat. No. 6, 180,370), Wright et al., (supra) and in the references cited therein, or in Gu et al. (1997, Thrombosis and Hematocyst 77(4):755-759). The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, such as an epitope on an antigen of interest, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to the humanized immunoglobulin coding sequences, including naturally-associated or heterologous promoter regions. The expression control sequences can be eukaryotic promoter systems in vectors capable of transforming or transfecting eukaryotic host cells or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference).

The invention also includes functional equivalents of the antibodies described herein. Functional equivalents have binding characteristics comparable to those of the antibodies, and include, for example, hybridized and single chain antibodies, as well as fragments thereof. Methods of producing such functional equivalents are disclosed in PCT Application WO 93/21319 and PCT Application WO 89/09622.

Functional equivalents include polypeptides with amino acid sequences substantially the same as the amino acid sequence of the variable or hypervariable regions of the antibodies. “Substantially the same” amino acid sequence is defined herein as a sequence with at least 70%, preferably at least about 80%, more preferably at least about 90%, even more preferably at least about 95%, and most preferably at least 99% homology to another amino acid sequence (or any integer in between 70 and 99), as determined by the FASTA search method in accordance with Pearson and Lipman, 1988 Proc. Nat'l. Acad. Sci. USA 85: 2444-2448. Chimeric or other hybrid antibodies have constant regions derived substantially or exclusively from human antibody constant regions and variable regions derived substantially or exclusively from the sequence of the variable region of a monoclonal antibody from each stable hybridoma.

Single chain antibodies (scFv) or Fv fragments are polypeptides that consist of the variable region of the heavy chain of the antibody linked to the variable region of the light chain, with or without an interconnecting linker. Thus, the Fv comprises an antibody combining site.

Functional equivalents of the antibodies of the invention further include fragments of antibodies that have the same, or substantially the same, binding characteristics to those of the whole antibody. Such fragments may contain one or both Fab fragments or the F(ab′)₂ fragment. The antibody fragments contain all six complement determining regions of the whole antibody, although fragments containing fewer than all of such regions, such as three, four or five complement determining regions, are also functional. The functional equivalents are members of the IgG immunoglobulin class and subclasses thereof, but may be or may combine with any one of the following immunoglobulin classes: IgM, IgA, IgD, or IgE, and subclasses thereof. Heavy chains of various subclasses, such as the IgG subclasses, are responsible for different effector functions and thus, by choosing the desired heavy chain constant region, hybrid antibodies with desired effector function are produced. Exemplary constant regions are gamma 1 (IgG1), gamma 2 (IgG2), gamma 3 (IgG3), and gamma 4 (IgG4). The light chain constant region can be of the kappa or lambda type.

The immunoglobulins of the present invention can be monovalent, divalent or polyvalent. Monovalent immunoglobulins are dimers (HL) formed of a hybrid heavy chain associated through disulfide bridges with a hybrid light chain. Divalent immunoglobulins are tetramers (H₂L₂) formed of two dimers associated through at least one disulfide bridge.

### Peptide Inhibitors

In one embodiment, the composition of the present invention comprises an isolated peptide inhibitor of PKCδ. In one embodiment, composition comprises an isolated PKCδ-derived peptide. The composition may comprise, for example, a fragment of PKCδ. In one embodiment, the composition comprises a fragment of PKCδ fused to, linked to or associated with HIV-1 Tat protein or a fragment thereof. In one embodiment, the inhibitor comprises a peptide derived from the first unique region (V1) of PKCδ (SFNSYELGSL (SEQ ID NO:1)) coupled via an N-terminal Cys-Cys bond to a membrane permeant peptide sequence in the HIV TAT gene product (YGRKKRRQRRR: (SEQ ID NO:2)) which forms a PKCδ-TAT inhibitor that effectively inhibits activation of PKCδ, but not other PKC isotypes.

The peptide of the present invention may be made using chemical methods. For example, peptides can be synthesized by solid phase techniques (Roberge J Y et al (1995) Science 269: 202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

The peptides of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The peptides of the invention may include unnatural amino acids formed by post-translational modification or by introducing unnatural amino acids during translation. A variety of approaches are available for introducing unnatural amino acids during protein translation.

A peptide or protein of the invention may be conjugated with other molecules, such as proteins, to prepare fusion proteins. This may be accomplished, for example, by the synthesis of N-terminal or C-terminal fusion proteins provided that the resulting fusion protein retains the functionality of inhibiting PKCδ.

A peptide or protein of the invention may be phosphorylated using conventional methods such as the method described in Reedijk et al. (The EMBO Journal 11(4):1365, 1992).

Cyclic derivatives of the peptides of the invention are also part of the present invention. Cyclization may allow the peptide to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The components that form the bonds may be side chains of amino acids, non-amino acid components or a combination of the two. In an embodiment of the invention, cyclic peptides may comprise a beta-turn in the right position. Beta-turns may be introduced into the peptides of the invention by adding the amino acids Pro-Gly at the right position.

It may be desirable to produce a cyclic peptide which is more flexible than the cyclic peptides containing peptide bond linkages as described above. A more flexible peptide may be prepared by introducing cysteines at the right and left position of the peptide and forming a disulphide bridge between the two cysteines. The two cysteines are arranged so as not to deform the beta-sheet and turn. The peptide is more flexible as a result of the length of the disulfide linkage and the smaller number of hydrogen bonds in the beta-sheet portion. The relative flexibility of a cyclic peptide can be determined by molecular dynamics simulations.

The peptides and fusion proteins of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

### Small Molecules

When the inhibitor of the invention is a small molecule, a small molecule inhibitor may be obtained using standard methods known to the skilled artisan. Such methods include chemical organic synthesis or biological means. Biological means include purification from a biological source, recombinant synthesis and in vitro translation systems, using methods well known in the art.

Combinatorial libraries of molecularly diverse chemical compounds potentially useful in treating a variety of diseases and conditions are well known in the art as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

### Genetic Modification

In other related aspects, the invention includes an isolated nucleic acid encoding an inhibitor of PKCδ, operably linked to a nucleic acid comprising a promoter/regulatory sequence such that the nucleic acid is preferably capable of directing expression of the protein encoded by the nucleic acid. Thus, the invention encompasses expression vectors and methods for the introduction of exogenous DNA into cells with concomitant expression of the exogenous DNA in the cells such as those described, for example, in Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York).

The desired polynucleotide can be cloned into a number of types of vectors. However, the present invention should not be construed to be limited to any particular vector. Instead, the present invention should be construed to encompass a wide plethora of vectors which are readily available and/or well-known in the art. For example, a desired polynucleotide of the invention can be cloned into a vector including, but not limited to a plasmid, a phagemid, a phage derivative, an animal viruse, and a cosmid. Vectors of particular interest include expression vectors, replication vectors, probe generation vectors, and sequencing vectors.

In specific embodiments, the expression vector is selected from the group consisting of a viral vector, a bacterial vector and a mammalian cell vector. Numerous expression vector systems exist that comprise at least a part or all of the compositions discussed above. Prokaryote- and/or eukaryote-vector based systems can be employed for use with the present invention to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al. (2001), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

For expression of the desired polynucleotide, at least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

Additional promoter elements, i.e., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either co-operatively or independently to activate transcription.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2001). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

A promoter sequence exemplified in the experimental examples presented herein is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, Moloney virus promoter, the avian leukemia virus promoter, Epstein-Barr virus immediate early promoter, Rous sarcoma virus promoter, as well as human gene promoters such as, but not limited to, the actin promoter, the myosin promoter, the hemoglobin promoter, and the muscle creatine promoter. Further, the invention should not be limited to the use of constitutive promoters. Inducible promoters are also contemplated as part of the invention. The use of an inducible promoter in the invention provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. Further, the invention includes the use of a tissue specific promoter, which promoter is active only in a desired tissue. Tissue specific promoters are well known in the art and include, but are not limited to, the HER-2 promoter and the PSA associated promoter sequences.

In order to assess the expression of the siRNA, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000 FEBS Lett. 479:79-82). Suitable expression systems are well known and may be prepared using well known techniques or obtained commercially. Internal deletion constructs may be generated using unique internal restriction sites or by partial digestion of non-unique restriction sites. Constructs may then be transfected into cells that display high levels of siRNA polynucleotide and/or polypeptide expression. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In the context of an expression vector, the vector can be readily introduced into a host cell, e.g., mammalian, bacterial, yeast or insect cell by any method in the art. For example, the expression vector can be transferred into a host cell by physical, chemical or biological means. It is readily understood that the introduction of the expression vector comprising the polynucleotide of the invention yields a silenced cell with respect to a regulator.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

Any DNA vector or delivery vehicle can be utilized to transfer the desired polynucleotide to a cell in vitro or in vivo. In the case where a non-viral delivery system is utilized, a preferred delivery vehicle is a liposome. The above-mentioned delivery systems and protocols therefore can be found in Gene Targeting Protocols, 2ed., pp 1-35 (2002) and Gene Transfer and Expression Protocols, Vol. 7, Murray ed., pp 81-89 (1991).

“Liposome” is a generic term encompassing a variety of single and multilamellar lipid vehicles formed by the generation of enclosed lipid bilayers or aggregates. Liposomes may be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). However, the present invention also encompasses compositions that have different structures in solution than the normal vesicular structure. For example, the lipids may assume a micellar structure or merely exist as nonuniform aggregates of lipid molecules. Also contemplated are lipofectamine-nucleic acid complexes.

### Methods of Treating Radiation Related Disorders

The invention includes methods for the treatment of a PKCδ related disorder. As used herein, the term “PKCδ related disorder” refers to any disease, disorder, or condition which is caused or characterized by activity of PKCδ, including SMN protein activity and mRNA metabolism. In one embodiment, the invention includes methods for the treatment of a disease or disorder associated with radiation.

Administration of a PKCδ inhibitor comprising one or more peptides, a small molecule, an antisense nucleic acid, a soluble receptor, or an antibody in a method of treatment can be achieved in a number of different ways, using methods known in the art.

It will be appreciated that a PKCδ inhibitor of the invention may be administered to a subject either alone, or in conjunction with another therapeutic agent. In one embodiment, an exogenous PKCδ inhibitor peptide is administered to a subject. The exogenous peptide may also be a hybrid or fusion protein to facilitate, for instance, delivery to target cells or efficacy. In one embodiment, a hybrid protein may comprise a tissue-specific targeting sequence.

The therapeutic and prophylactic methods of the invention thus encompass the use of pharmaceutical compositions comprising a PKCδ inhibitory peptide, fusion protein, small molecule, soluble receptor, or antibody of the invention and/or an isolated nucleic acid encoding a PKCδ inhibitory peptide, fusion protein small molecule, soluble receptor, or antibody of the invention to practice the methods of the invention. The pharmaceutical compositions useful for practicing the invention may be administered to deliver a dose of from ng/kg/day and 100 mg/kg/day. In one embodiment, the invention envisions administration of a dose which results in a concentration of the compound of the present invention from 1 μM and 10 μM in a mammal.

Typically, dosages which may be administered in a method of the invention to a mammal, preferably a human, range in amount from 0.5 μg to about 50 mg per kilogram of body weight of the mammal. While the precise dosage administered will vary depending upon any number of factors, including but not limited to, the type of mammal and type of disease state being treated, the age of the mammal and the route of administration. Preferably, the dosage of the compound will vary from about 1 μg to about 10 mg per kilogram of body weight of the mammal. More preferably, the dosage will vary from about 3 μg to about 1 mg per kilogram of body weight of the mammal.

The compound may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

In a preferred embodiment, the invention includes methods for treating or preventing a disease or disorder associated with radiation by inhibiting the activity of PKCδ. In one aspect, PKCδ activity is inhibited by administering a PKCδ inhibitor to a subject in order to inhibit the degradation of a protein. In one embodiment, a PKCδ inhibitor is administered to a subject to inhibit the degradation of an SMN protein. In another aspect, PKCδ activity may be inhibited by providing exogenous SMNΔ7^{S270A}.

The administration of the polypeptide of the invention to the subject having a PKCδ related disorder may be accomplished using gene therapy. Gene therapy, which is based on inserting a therapeutic gene into a cell by means of an ex vivo or an in vivo technique. Suitable vectors and methods have been described for genetic therapy in vitro or in vivo, and are known as expert on the matter; see, for example, Giordano, Nature Medicine 2 (1996), 534-539; Schaper, Circ. Res 79 (1996), 911-919; Anderson, Science 256 (1992), 808-813; Isner, Lancet 348 (1996), 370-374; Muhlhauser, Circ. Res 77 (1995), 1077-1086; Wang, Nature Medicine 2 (1996), 714-716; WO94/29469; WO97/00957 or Schaper, Current Opinion in Biotechnology 7 (1996), 635-640 and the references quoted therein. The polynucleotide codifying the polypeptide of the invention can be designed for direct insertion or by insertion through liposomes or viral vectors (for example, adenoviral or retroviral vectors) in the cell. Preferably the cell is a cell of the germinal line, an embryonic cell or egg cell or derived from the same, more preferably the cell is a core cell. Suitable gene distribution systems that can be used according to the invention may include liposomes, distribution systems mediated by receptor, naked DNA and viral vectors such as the herpes virus, the retrovirus, the adenovirus and adeno-associated viruses, among others. The distribution of nucleic acids to a specific site in the body for genetic therapy can also be achieved by using a biolistic distribution system, such as that described by Williams (Proc. Natl. Acad. Sci. USA, 88 (1991), 2726-2729). The standard methods for transfecting cells with recombining DNA are well known by an expert on the subject of molecular biology, see, for example, WO94/29469; see also supra. Genetic therapy can be carried out by directly administering the recombining DNA molecule or the vector of the invention to a patient or transfecting the cells with the polynucleotide or the vector of the invention ex vivo and administering the transfected cells to the patient.

### Pharmaceutical Compositions

The present invention also provides pharmaceutical compositions comprising one or more of the compositions described herein. Formulations may be employed in admixtures with conventional excipients, i.e., pharmaceutically acceptable organic or inorganic carrier substances suitable for administration to the wound or treatment site. The pharmaceutical compositions may be sterilized and if desired mixed with auxiliary agents, e.g., lubricants, preservatives, stabilizers, wetting agents, emulsifiers, salts for influencing osmotic pressure buffers, coloring, and/or aromatic substances and the like. They may also be combined where desired with other active agents, e.g., other analgesic agents.

Administration of the compositions of this invention may be carried out, for example, by parenteral, by intravenous, intratumoral, subcutaneous, intramuscular, or intraperitoneal injection, or by infusion or by any other acceptable systemic method.

As used herein, “additional ingredients” include, but are not limited to, one or more of the following: excipients; surface active agents; dispersing agents; inert diluents; granulating and disintegrating agents; binding agents; lubricating agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

In an embodiment, the composition includes an anti-oxidant and a chelating agent that inhibits the degradation of one or more components of the composition. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition that may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Other active agents useful in the treatment of fibrosis include anti-inflammatories, including corticosteroids, and immunosuppressants.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intrasternal injection, intratumoral, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, Pa.), which is incorporated herein by reference.

Liquid suspensions may be prepared using conventional methods to achieve suspension of the composition of the invention in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para- hydroxybenzoates, ascorbic acid, and sorbic acid.

### Treatment Methods

The present invention provides a method for the treatment or prevention of radiation induced injury, or a disease or disorder associated therewith, in a subject in need thereof. In certain embodiments, the method comprises administering an effective amount of a composition described herein to a subject diagnosed with, suspected of having, or at risk for developing radiation induced injury. In one embodiment, the composition is administered locally to a cell, tissue or organ of a subject. In one embodiment, the composition is administered systemically to the subject.

The composition of the invention may be administered to a patient or subject in need in a wide variety of ways. Modes of administration include intraoperatively intravenous, intravascular, intramuscular, subcutaneous, intracerebral, intraperitoneal, soft tissue injection, surgical placement, arthroscopic placement, and percutaneous insertion, e.g., direct injection, cannulation or catheterization. Any administration may be a single application of a composition of invention or multiple applications. Administrations may be to single site or to more than one site in the individual to be treated. Multiple administrations may occur essentially at the same time or separated in time.

In certain embodiments, the composition of the invention is administered during or following a radiation-based therapy. For example, in subjects undergoing whole body radiation therapy for the treatment of cancer.

Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions of the present invention may be administered in a manner appropriate to the disease to be treated (or prevented). The quantity and frequency of administration will be determined by such factors as the condition of the subject, and the type and severity of the subject's disease, although appropriate dosages may be determined by clinical trials.

When “therapeutic amount” is indicated, the precise amount of the compositions of the present invention to be administered can be determined by a physician with consideration of individual differences in age, weight, disease type, extent of disease, and condition of the patient (subject).

The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the compositions of the present invention are administered to a patient by intradermal or subcutaneous injection. In another embodiment, the compositions of the present invention are preferably administered by i.v. injection.

### Kits

The invention also includes a kit comprising a PKCδ inhibitor and an instructional material which describes, for instance, administering the PKCδ inhibitor to a subject as a prophylactic or therapeutic treatment as described elsewhere herein. In an embodiment, the kit further comprises a (preferably sterile) pharmaceutically acceptable carrier suitable for dissolving or suspending the therapeutic composition, comprising a PKCδ inhibitor, for instance, prior to administering the inhibitor to a subject. Optionally, the kit comprises an applicator for administering the inhibitor. In one embodiment of the invention, the applicator is designed for pulmonary administration of the PKCδ inhibitor. In another embodiment, the kit comprises an antibody that specifically binds an epitope on PKCδ. Preferably, the antibody recognizes a human PKCδ.

A kit comprising a nucleic acid encoding a peptide or antibody of the invention and an instructional material is also provided.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1: PKCδ Inhibition as a Novel Medical Countermeasure for Radiation-Induced Vascular Damage

Interaction of the immune system and endothelial barrier plays an important role in radiation induced damage through the release of reactive species and cytokines at different time points post-IR (Williams and McBride, 2011, Int J Radiat Biol, 87(8):851-868; Chiang et al., Int. J. Radiat. Biol, 72, 45-53; Schaue et al., 2015, Semin Radiat Oncol, 25:4-10). While recombinant growth factors such as recombinant granulocyte colony-stimulating factor (G-CSF or Neupogen) and recombinant granulocyte-macrophage colony-stimulating factor (GM-CSF or Neulasta) have been recently approved by the FDA for treatment of hematopoietic acute radiation syndrome, their effectiveness depends on “highly trigger-based supportive care” (Singh and Seed, 2017, Int J Radiat Biol, 1-19; Farese and MacVittie, 2015, Drugs Today (Barc), 51:537-548) and do not address the IR damage to the endothelium (Hofer et al., 2014, Molecules, 19:4770-4778; Singh et al., 2015, Cytokine 71, 22-37). Hence, there is a need for discovery and development of new radiomitigators and radiation medical countermeasures.

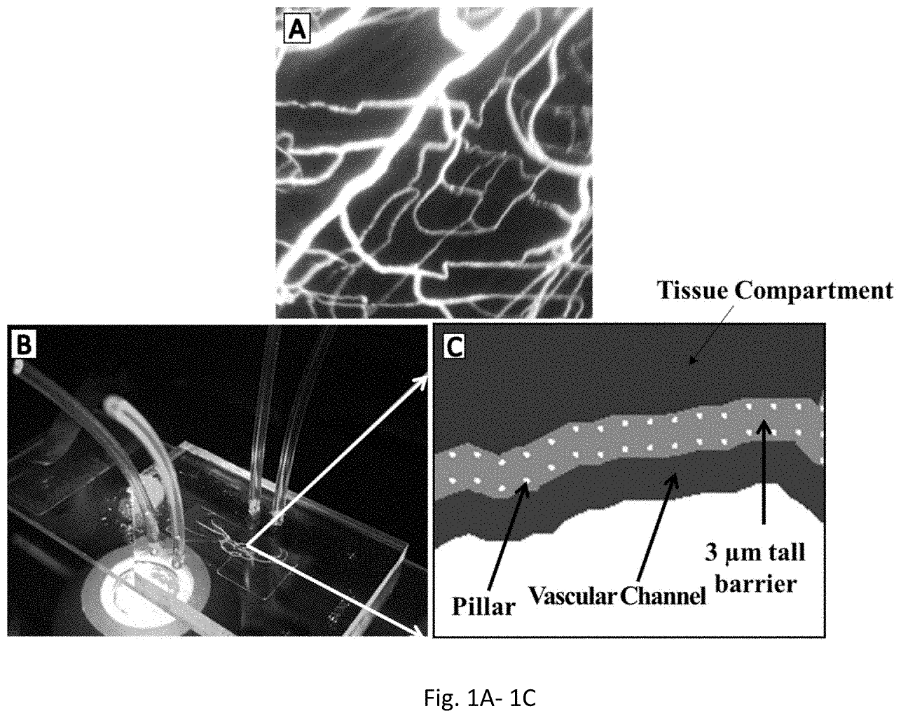

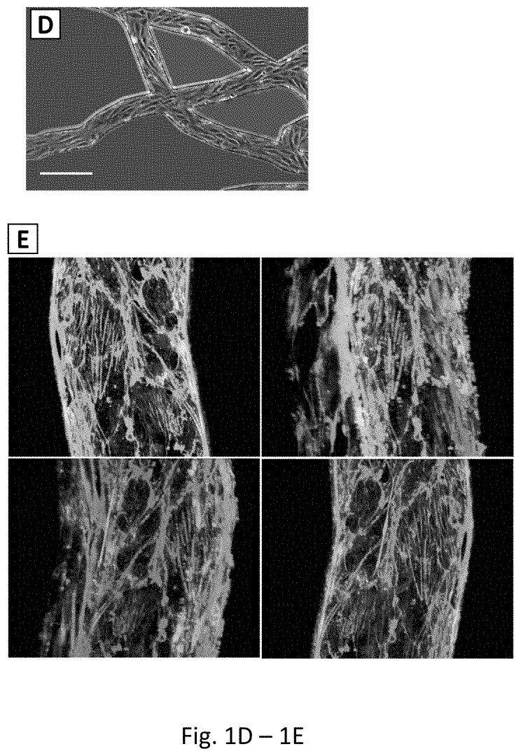

Recent studies indicate a role for PKCδ in radiation-induced apoptosis (Yuan et al., 1998, Oncogene, 16:1643-1648; Wie et al., 2014, J Biol Chem, 289:10900-10908; Mitsutake et al., 2001, Oncogene 20, 989-996), radiation-induced cell proliferation anomaly (Lee et al., 2002, Cell Growth and Differentiation, 13:237-246), and radiation-induced tissue damage in salivary gland, thymus (Humphries et al., 2006, Journal of Biological Chemistry, 281(14):9728-9737; Reyland and Jones, 2016, Pharmacology & Therapeutics, 165:1-13), and thyroid cells (Reyland and Jones, 2016, Pharmacology & Therapeutics, 165:1-13; Wie et al., 2014, Journal of Biological Chemistry, 289(15):10900-10908; Mitsutake et al., 2001, Oncogene, 20(8):989-996). However, less is known about how PKCδ is activated in endothelium following radiation exposure or its role in neutrophil-endothelial cell interaction and endothelial cell activation, in part due to the lack of realistic fluidic models for in vitro reconstitution of disease-related cell types and tissues (Seok et al., 2013, Proceedings of the National Academy of Sciences, 110(9):3507-3512). In this study, a novel biomimetic microfluidic assay (bMFA), a physiologically relevant in vitro environment (Prabhakarpandian et al., 2011, Microvasc Res, 82:210-220; Prabhakarpandian et al., 2008, Biomed Microdevices, 10:585-595; Rosano et al., 2009, Biomed Microdevices, 11:1051-1057; Lamberti et al., 2014, AnalChem, 86(16):8344-8351), was used to discover mechanisms by which PKCδ impacts neutrophil-endothelial cell interaction during radiation-induced inflammation and to show that a PKCδ-TAT peptide inhibitor can significantly downregulate the increased neutrophil-endothelial cell interaction post-IR. It is demonstrated that endothelial cells form a 3D lumen in the bMFA to provide a physiologically realistic environment to study cell-cell interactions (FIG. 1). The results presented herein further demonstrate that PKCδ inhibition in irradiated endothelial cells is a potent downregulator of leukocyte-endothelial interaction, protects endothelial barrier integrity, and reduces endothelial permeability to control level even when the inhibitor is administered 24 hours post radiation exposure.

A role for PKCδ in inflammation-induced neutrophil-endothelial cell interaction under both static and flow conditions has been demonstrated (Mondrinos et al., 2014, The American Journal of Pathology, 184(1):200-213; Soroush et al., 2016, Journal of Leukocyte Biology, 100(5):1027-1035). Neutrophils rolling and adhesion on endothelial cells is mediated by selectins (e.g. E- and P-selectin) and integrins (e.g. ICAM-1), while the transition to migration is mediated by ICAM-1 and VCAM-1 (Springer, 1994, Cell, 76:301-314; Ley et al., 2007, Nat Rev Immunol, 7:678-689; Springer, 1990, Scand J Immunol, 32:211-216). A number of molecules involved in the leukocyte adhesion cascade are also involved in radiation-induced tissue damage. For example, intravital microscopy demonstrated that adhesion molecules (e.g. ICAM-1) are upregulated in irradiated tissue in vivo and the resulting increase in leukocyte adhesion could be modulated with administration of an anti-inflammatory agent (Dexamethasone) or an anti-ICAM-1 antibody (Yuan et al., 2005, Radiat. Res, 163:544-551; Prabhakarpandian et al., 2001, Microcirc, 8:355-364; Yuan et al., 2003, Brain Res, 969:59-69). In this study, the physiologically realistic, 3D cell culture environment of bMFA was used to show that radiation exposure increases neutrophil adhesion and migration across endothelial cells at all radiation doses studied. Interestingly, the increase in neutrophils migration levels was less pronounced at the 5 Gy dose as compared to lower doses and may be a result of decreased PKCδ activation at this radiation dose. However, this dose-dependent difference was not observed in neutrophil adhesion levels suggesting differential regulation by PKCδ. This differential impact of radiation on neutrophil migration vs. adhesion indicates that not all adhesion molecules are uniformly impacted by ionizing radiation. Further studies of neutrophil-endothelial cell interaction and adhesion molecule upregulation under shear flow may be required to better understand the role of ionizing radiation in radiation induced inflammatory process (Panes et al., 1995, Gastroenterology, 108:1761-1769; Ansari et al., 2007, Radiat. Res, 167:80-86; Panes and Granger, 1998, Gastroenterology, 114:1066-1090; Micke et al., 2005, Strahlentherapie and Onkologie : Organ der Deutschen Rontgengesellschaft . . . [et al], 181:313- 319).

Consistent with previous reports (Tousi et al., 2010, Microvasc Res, 80(3):384-388; Soroush et al., 2016, Journal of Leukocyte Biology, 100(5):1027-1035; Prabhakarpandian et al., 2011, Microcirculation, 18:380-389), neutrophil adhesion, as well as its downregulation by PKCδ inhibition, was most pronounced in vessels with low shear flows and near bifurcations. Treatment with the PKCδ inhibitor, even 24 hours post-IR, decreases expression of VCAM-1, ICAM-1, P-selectin, but not E-selectin. This decreased expression of adhesion molecules was associated with decreased permeability and decreased neutrophil adherence and migration through irradiated endothelial cells in response to fMLP.

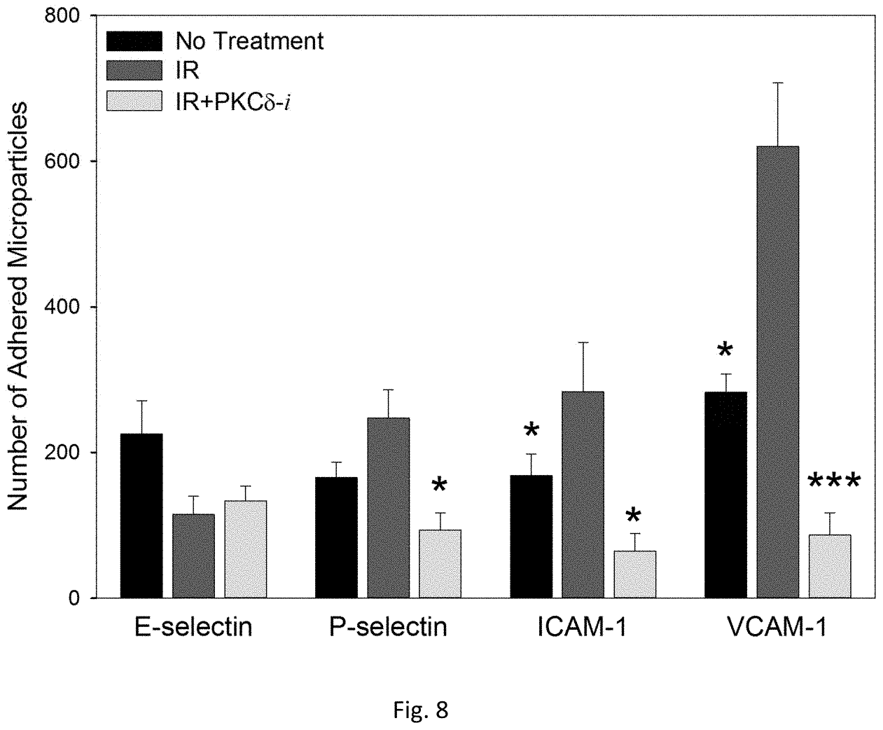

While a role for PKCδ in radiation injury has been established in several cell types (Mitsutake et al., 2001, Oncogene, 20:989-996; Wie et al., 2014, Journal of Biological Chemistry, 289:10900-10908; Reyland and Jones, 2016, Pharmacology & Therapeutics, 165:1-13), the exact role of PKCδ in regulating radiation-induced adhesion molecule expression in endothelial cells is not well established. In endothelial cells, PKCδ is involved in NF-κB activation, adhesion molecule expression, and production of inflammatory mediators important in neutrophil transmigration (Cummings et al., 2004, J Biol Chem 279, 41085-41094; Page et al., 2003, J Immunol 170, 5681-5689; Rahman et al., 2001, Mol Cell Biol 21, 5554-5565; Woo et al., 2005, Am J Physiol Lung Cell Mol Physiol 288, L307-316; Mondrinos et al., 2014, The American Journal of Pathology 184, 200-213). In vivo, systemic inflammation (e.g. induced by sepsis) produced increased expression of ICAM-1 which was attenuated by treatment with the PKCδ-TAT inhibitor (Mondrinos et al., 2014, The American Journal of Pathology, 184:200-213; Mondrinos et al., 2015, Journal of Pharmacology and Experimental Therapeutics, 355:86-98). In vitro mechanistic studies demonstrated PKCδ regulated adhesion molecule expression in human large vessel endothelial (HUVEC) and microvascular endothelial cells (human pulmonary microvascular endothelial cells (PMVEC)) (Soroush et al., 2016, Journal of Leukocyte Biology, 100:1027-1035; Mondrinos et al., 2014, The American Journal of Pathology 184, 200-213). These findings (FIG. 8) support the hypothesis that PKCδ regulates these key components which are critical to vascular endothelial cell activation after radiation exposure.

In summary, a novel biomimetic microfluidic assay (bMFA) was used to study the role of PKCδ as a regulator of human neutrophil-endothelial cell interaction and endothelium integrity post radiation exposure. The findings presented herein indicate a key role for PKCδ regulation of radiation-induced changes in endothelial cell barrier structure and function, expression of several key cell adhesion molecules, leukocyte-endothelial cell interaction and leukocyte migration through endothelium. Furthermore, the findings indicate that PKCδ-TAT peptide inhibitor can significantly downregulate the increased neutrophil-endothelial cell interaction and preserve endothelial cell integrity post-IR. Without being bound by theory, it is proposed that PKCδ inhibition may serve as a novel medical countermeasure for treating radiation-induced vascular damage. The novel biomimetic microfluidic assay (bMFA) provides a tool for rapid screening of novel therapeutics for treating radiation injury.

The materials and methods used for these experiments are now described

### Materials, Equipment, and Reagents

A mouse monoclonal anti-human ICAM-1, a mouse monoclonal anti-human VCAM-1, a mouse monoclonal anti-human E-selectin, a mouse monoclonal anti-human VE-cadherin, and an Alexa fluor 594 goat anti-mouse IgG were purchased from Santa Cruz Biotechnology (Dallas, Tex.). A mouse monoclonal anti-human P-selectin was purchased from Abcam PLC (Cambridge, Mass.). Human fibronectin was obtained from BD Biosciences (San Jose, Calif.). Protein A was purchased from Thermo Scientific (Waltham, Mass.). Fluorescent 9.9 μm microparticles (green: excitation 468 nm, emission 508 nm) were purchased from Duke Scientific (Palo Alto, Calif.). Human umbilical vein endothelial cells (HUVEC) were purchased from Lonza Walkersville (Walkersville, Md.). Carboxyfluorescein diacetate succinimidyl ester (CFDA/SE) probe from Molecular Probes (Carlsbad, Calif.), Bovine Serum Albumin (BSA) was purchased from Sigma-Aldrich, Hanks' Balanced Salt Solution (HBSS), Trypsin/EDTA, Formalin, Triton X-100, Draq5, 40 kDa Texas Red conjugated dextran, and Hoechst 33342 from Thermofisher Scientific (Rockford, Ill.), and Alexa Fluor® 488 Phalloindin from Life Technologies Corporation (Carlsbad, Calif.).

A Nikon TE200 fluorescence microscope equipped with an automated stage was used for performing experiments. An Olympus FluoView FV1000 confocal microscope equipped with a fully automated stage was used for capturing confocal image stacks. Images were acquired using an ORCA Flash 4 camera (Hamamatsu Corp., USA). PhD Ultra Syringe pump (Harvard Apparatus) was used for injecting media, permeability dye, or neutrophil suspension to the bMFA with high precision. A stage warmer was used to keep the bMFA at 37° C. NIS Elements software (Nikon Instruments Inc., Melville, N.Y.) was used to control the microscope stage and the camera.

### PKCδ Inhibitor Peptide Synthesis

PKCδ activity was selectively inhibited by a peptide antagonist that consisted of a peptide derived from the first unique region (V1) of PKCδ (SFNSYELGSL: amino acids 8-17) coupled via an N-terminal Cys-Cys bond to a membrane permeant peptide sequence in the HIV TAT gene product (YGRKKRRQRRR: amino acids 47-57 of TAT) (Chen et al., 2001, Proc Natl Acad Sci USA, 98(20):11114-11119). The PKCδ TAT peptide produces a unique dominant-negative phenotype that effectively inhibits activation of PKCδ but not other PKC isotypes. The peptide was synthesized by Mimotopes (Melbourne, Australia) and purified to >95% by HPLC.

### Design and Fabrication of the Microfluidic Assay

Methods for design and fabrication of a microfluidic assay and in vivo validation have been published (Prabhakarpandian et al., 2008, Biomed Microdevices, 10:585-595; Soroush et al., 2016, Journal of Leukocyte Biology, 100(5):1027-1035; Lamberti et al., 2014, AnalChem, 86(16):8344-8351). Briefly, a modified Geographic Information System (GIS) approach was used to digitize the in vivo microvascular networks (FIG. 1A) which were lithographically patterned on polydimethylsiloxane (PDMS) (FIG. 1B). Microfabricated pillars (10 μm diameter) were used to fabricate the 3×100 μm pores resulting in a network of vascular channels connected to a tissue compartment via a 3 μm porous barrier (FIG. 1C), which is the optimum size for neutrophil migration.

### Seeding of Endothelial Cells in bMFA

Endothelial cells (human umbilical vein endothelial cells (HUVEC)) were cultured in growth media (EGM, Genlantis® PrimaPure®) and used between passages 3-6. The bMFA was coated with fibronectin and endothelial cells were cultured under shear flow (inlet flow rate of 0.5 μl/min) for 24 hours (Lamberti et al., 2014, AnalChem, 86(16):8344-8351). Consistent with published data (Soroush et al., 2016, Journal of Leukocyte Biology, 100(5):1027-1035; Deosarkar et al., 2015, PLoS One, 10(11):e0142725; Tang et al., 2017, Sci Rep, 7), endothelial cells in bMFA form a confluent lumen and aligned in the direction of flow (FIG. 1D). Formation of the 3D lumen in vascular channels under physiological conditions was confirmed using confocal microscopy (FIG. 1E) (Soroush et al., 2016, Journal of Leukocyte Biology, 100(5):1027-1035; Deosarkar et al., 2015, PLoS One, 10(11):e0142725). In agreement with the observed complexity of in vivo flow conditions (Rosano et al., 2009, Biomed Microdevices, 11:1051-1057), shear stress is different in different vessels of the bMFA due to flow distribution in the complex in vivo-like geometry. However, by keeping inlet flow conditions constant, shear stress in a given vessel in bMFA is the same across different measurements. Assays in which neutrophils freely entered the tissue compartment without attachment were discarded.

### Ionizing Radiation Treatment of Endothelial Cells

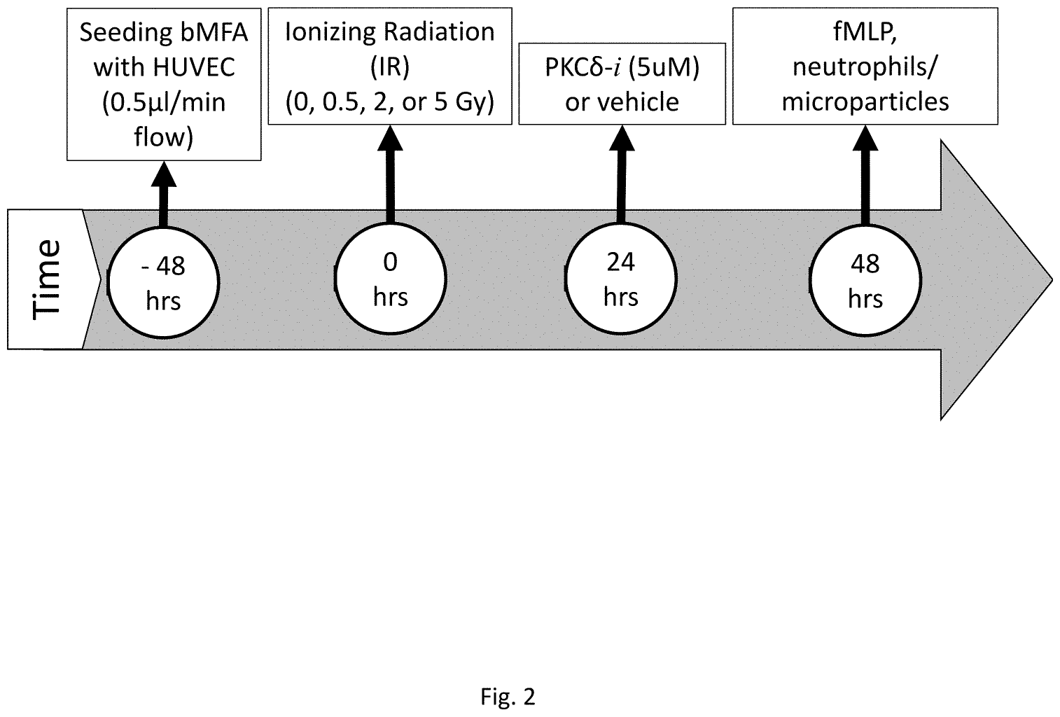

Endothelial cells in the bMFA were exposed to 0 Gy (no treatment), 0.5 Gy, 2 Gy, or 5 Gy (1.13 Gy per minute) of radiation treatment from an X-RAD 320 irradiator. At 24 hours after ionizing irradiation (post-IR) treatment, EGM or EGM containing the PKCδ-TAT peptide inhibitor (PKCδ-i) (5 μM) was injected into the bMFA. This treatment timepoint (e.g. 24 hours post-IR) was selected to approximate clinically relevant treatment scenarios following radiological disasters (Maxwell, 1982, Am J Public Health, 72:275-279). At 48 hours post-IR, the tissue compartment was filled with EGM containing a chemoattractant (fMLP, 1 μM) or EGM (control) before introducing neutrophils or antibody coated microparticles into the vascular compartment (FIG. 2).

### PKCδ Phosphorylation and Translocation

HUVEC cells grown in 6 well plates to confluency were exposed to 0 Gy (no treatment), 0.5 Gy, 2 Gy, or 5 Gy (1.13 Gy per minute) of radiation treatment and then incubated for one hour at 37° C. The cells were placed on ice, harvested, and the membrane and cytoplasm fractions were isolated according to the manufacturer's instructions using a Subcellular Protein Fractionation Kit for Cells (Thermo Fisher Scientific, Watham, Mass.). Samples for Western blot analysis were prepared by mixing an aliquot of the samples with 2× sample buffer and heating for 5 minutes at 95° C. Purity of membrane fractions was routinely determined by probing fractions for cytoplasmic (GAPDH) and membrane (VE-cadherin) markers. Proteins (30 μg/lane) were separated on 4-12% SDS-PAGE gels and transferred to nitrocellulose membranes as described previously. Translocation of phosphorylated PKCδ was determined by immunoblotting using a phospho-specific PKCδ (Ser643/676) antibody (Cell Signaling Technology, Beverly, Mass., USA) as described previously (Kilpatrick et al., 2010, J Leukoc Biol, 87(1):153-164; Begley et al., 2004, Biochem Biophys Res Commun, 318:949-954; Vary et al., 2005, Am J Physiol Endocrinol Metab, 289:E684-694). Translocation of PKCδ to the membrane (particulate fraction) was quantitated by densitometry analysis of Western blots with ImageJ software version 1.46r (National Institutes of Health), and the values were expressed in arbitrary densitometry units.

### Neutrophil Isolation and Labeling

Heparinized human blood was obtained from healthy adult donors. Human neutrophils were isolated using ficoll-hypaque separation, dextran sedimentation, and hypotonic lysis to remove erythrocytes (Kilpatrick et al., 2010, J Leukoc Biol, 87(1):153-164; Mondrinos et al., 2014, The American Journal of Pathology, 184(1):200-213). After isolation, neutrophils were counted, suspended in HBSS (5×10⁶ cells/mL), and labeled using CFDA/SE probe for 10 minutes at room temperature. Neutrophils were introduced into the vascular channels of the bMFA at a flow rate of 1 μl/min.

### Preparation of Antibody Coated Microparticles

The level of adhesion of antibody (e.g. anti-ICAM-1) coated microparticles to endothelial cells was used as an index of the level of upregulation of adhesion molecules post-IR (2 Gy). Briefly, 9.9 μm fluorescent polystyrene microparticles were washed with a sodium bicarbonate buffer and coated with protein A (300 μg/ml) via passive adsorption and incubated overnight at room temperature. Microparticles were then washed and incubated in a blocking buffer (1% BSA in HBSS) at room temperature. Microparticles (5 million particles/ml) were counted, diluted in blocking buffer, and incubated with antibodies to ICAM-1, VCAM-1, P-selectin, or E-selectin for 30 min. Antibody coated microparticles were then suspended in EGM and introduced into the bMFA as described before (Soroush et al., 2016, Journal of Leukocyte Biology, 100(5):1027-1035). The level of adhesion of a given antibody (e.g. anti-ICAM-1) coated microparticles to endothelial cells was used as an index of the level of upregulation of that adhesion molecule (Kiani et al., 2002, Pharm Res, 19(9):1317-1322).

### Permeability Measurements

The vascular compartment was connected to a Hamilton gas tight syringe filled with Texas Red 40 kDa dextran (25 μM in EGM) mounted on a syringe pump. Permeability was measured by imaging the bMFA every minute for 2 hrs while the dextran solution flowed through the vascular channels (flow rate 1 μl/min). Using a previously published method (Deosarkar et al., 2015, PLoS One, 10(11):e0142725), the following equation was used to calculate permeability (P) of dextran across the endothelium in bMFA:

$$P = \frac{1}{{I}_{{v}_{0}}} \frac{V}{S} \frac{{dI}_{t}}{dt} , \qquad ( 1 )$$

where Iₜ is the average intensity in the tissue compartment, I_{V}_{_{Q}} is the maximum fluorescence intensity of the vascular channel, V/S is the ratio of vascular channel volume to its surface area.

### Immunofluorescence Staining

The formation of endothelial cell-cell adherens junctions in microvessels of the bMFA was characterized using immunostaining against VE-cadherin (Tang et al., 2017, Sci Rep, 7). To study morphological changes in cells post-IR, actin filaments were stained with phalloidin and cell nucleus were stained with Hoechst 33342.

### Data Analysis

Cells that did not move for 30 seconds were considered adherent. Adhesion level of neutrophils to the endothelium reached steady state after 10 minutes of flow and was quantified by scanning the entire network. The number of migrated neutrophils was quantified using time-lapse imaging every 3 minutes for 60 min. Nikon Elements and Fiji software were used to collect and analyze the data (Schindelin et al., 2012, Nat Methods, 9:676-682). Data are presented as Mean±SEM. Statistical significance were determined by student t-test, one-way or two-way analysis of variance (ANOVA) using SigmaPlot software. Differences were considered statistically significant if p<0.05.

The results of the experiments are now described.

### Radiation Activates PKCδ in Endothelial Cells

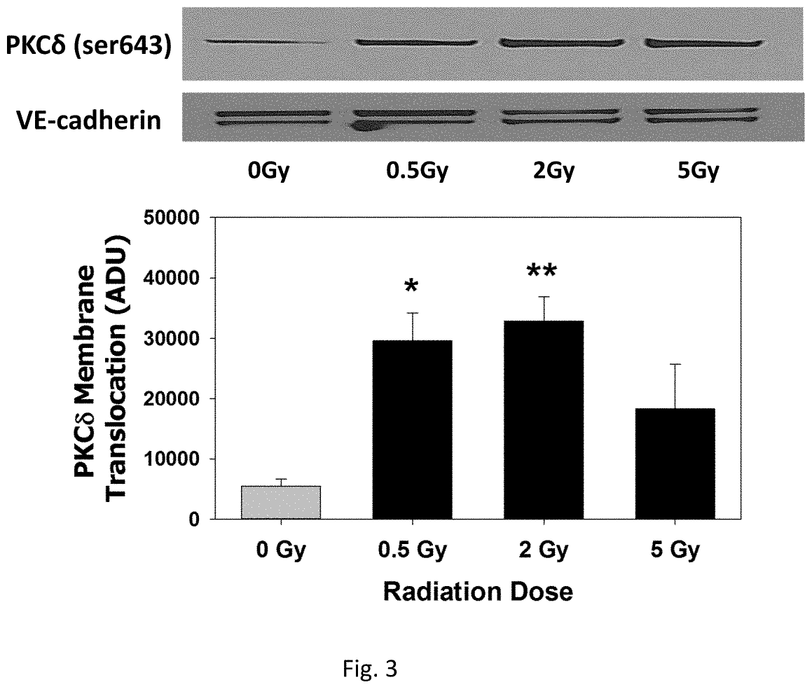

Activation of PKCδ is a multistep process that requires both phosphorylation and translocation of PKCδ from the cytosol to the membranous sites. Phosphorylation of PKCδ on Thr505 leads to an autophosphorylation step and phosphorylation on Ser643 in the PKCδ activation loop (Kilpatrick et al., 2010, J Leukoc Biol, 87(1):153-164; Vary et al., 2005, Am J Physiol Endocrinol Metab, 289:E684-694; Le Good et al., 1998, Science, 281:2042-2045), a site that regulates enzymatic activity and protein: protein interactions (Steinberg, 2004, Biochem J, 384:449-459). As shown in FIG. 3, there is little phosphorylated PKCδ present in membrane fractions of control HUVEC (0 Gy). In contrast, exposure to IR results in significant increase in translocation of phosphorylated PKCδ exposed to 0.5 and 2Gy. PKCδ translocation in response to 5Gy IR was variable and did not reach statistical significance. Thus, irradiation induces phosphorylation and translocation of PKCδ in endothelial cells.

### PKCδ Inhibition Preserves Integrity of Irradiated Endothelial Cells

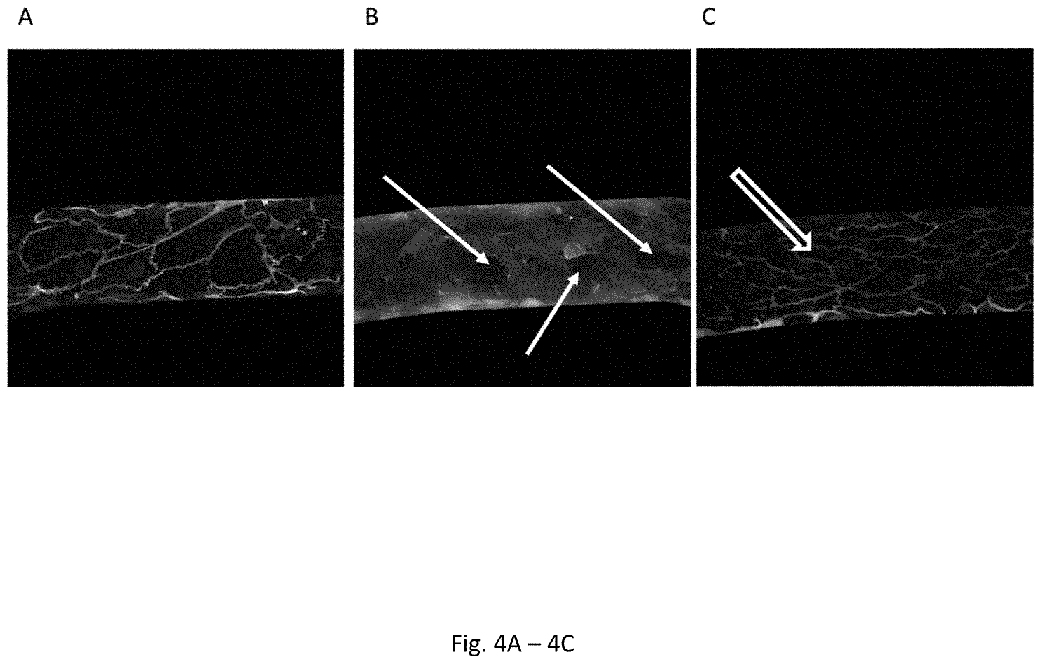

Endothelial cells formed a complete lumen and aligned in the direction of flow (FIG. 4A). Irradiated endothelial cells in the bMFA showed significant changes in morphology, decreased expression of F-actin filaments, lack of F-actin alignment with flow direction, and decreased adherens junctions expression 48 hours post-IR, indicating damage to the endothelial barrier integrity (FIG. 4B). At 24 hours post-IR, treatment with the inhibitor for 24 hours attenuated this damage as indicated by alignment of F-actin filaments with flow and stronger VE-cadherin expression (FIG. 4C).

### PKCδ Inhibition Modulates the Increase in Permeability of Irradiated Endothelial Cells

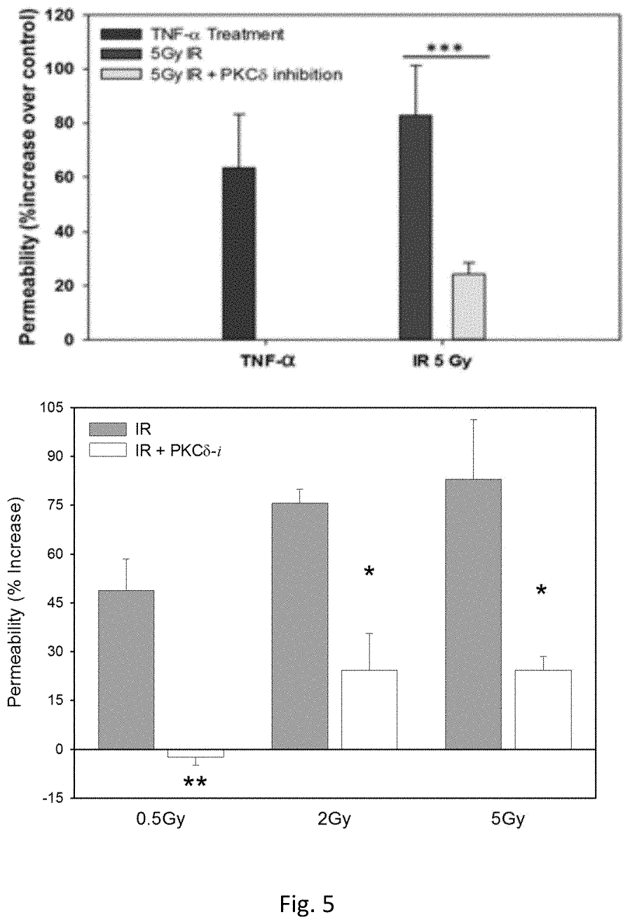

Integrity of endothelial cell barrier post-IR was directly assessed in the bMFA by measuring the dextran permeation from the vascular channels to the tissue compartment. Permeability rates across the endothelial barrier were measured 48 hours post-IR±the PKCδ inhibitor administered at 24 hours post-IR. Exposure to 0.5 Gy, 2 Gy, or 5 Gy IR significantly increased dextran permeability from control levels to 49-83% at 48 hours post-IR exposure (FIG. 5). Treatment of cells with the PKCδ inhibitor for 24 hours significantly reduced permeability, back to control levels for 0.5 Gy IR treatment, while reducing it by 67% and 70% for 2 Gy and 5 Gy IR exposure, respectively.

### PKCδ Inhibition Attenuates Neutrophil Migration Following Endothelial Cell Irradiation

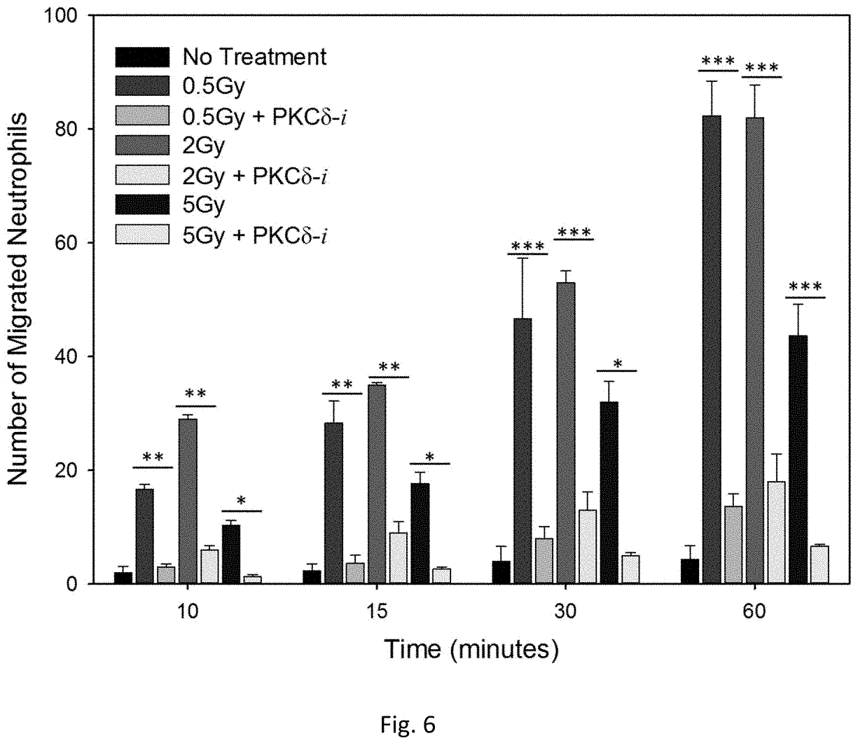

Neutrophil migration across endothelium into the tissue compartment was used to further assess endothelial barrier function after irradiation. In bMFA, neutrophil migration across irradiated endothelial cells in response to fMLP significantly increased over 60 minutes at each dose of radiation (FIG. 6). Of interest, the increase in neutrophil migration after 5 Gy IR treatment was significantly less pronounced as compared to other irradiated groups. Neutrophil migration across IR treated endothelial cells was significantly reduced after inhibition of PKCδ by 84%, 78%, and 86% for IR doses of 0.5 Gy, 2 Gy, and 5 Gy, respectively (FIG. 6). Thus, PKCδ inhibition attenuated neutrophil migration. Furthermore, the PKCδ inhibitor was able to significantly decrease neutrophil migration even at 5 Gy when neutrophil migration was reduced as compared to lower doses of radiation.

### PKCδ Inhibition Attenuates Neutrophil Adhesion to Irradiated Endothelium

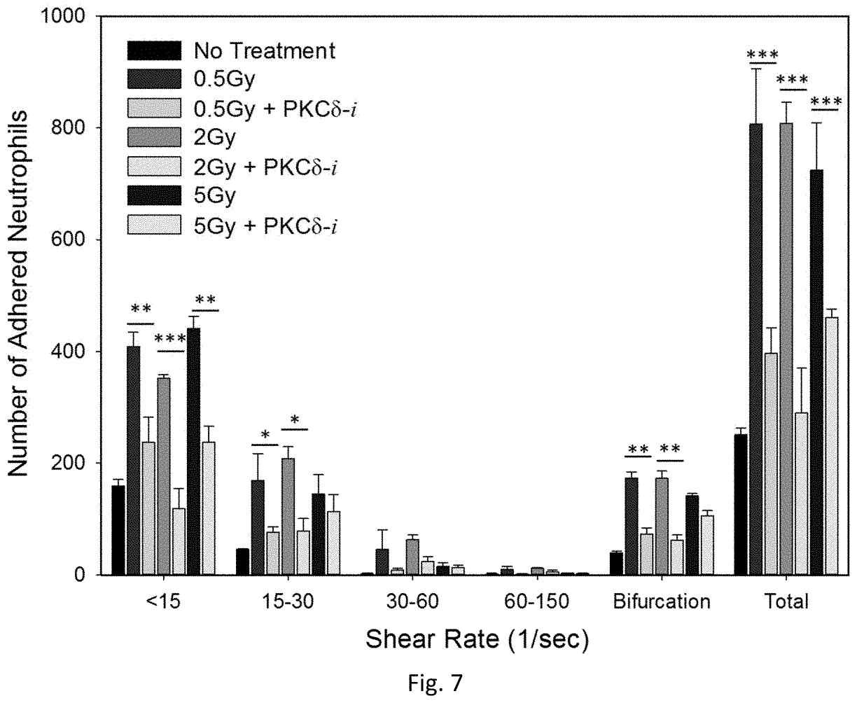

To further explore the effect of PKCδ inhibition on neutrophil-endothelial cell interaction post-IR, neutrophil adhesion to endothelial cells under shear flow conditions was investigated. IR treatment significantly increased neutrophil adhesion to endothelial cells as compared to controls with no significant differences between 0.5 Gy, 2 Gy, and 5 Gy IR treatment groups (FIG. 7). PKCδ inhibition significantly reduced the total number of adhered neutrophils by 51%, 64%, and 36% for IR doses of 0.5 Gy, 2 Gy, and 5 Gy, respectively. The reduction in neutrophil adhesion after PKCδ inhibition was most pronounced in vessels with low shear flows and near bifurcations. These findings indicate that PKCδ inhibition significantly reduces neutrophil adhesion to irradiated endothelial cells.

### PKCδ Inhibition Downregulates Expression of P-selectin, VCAM-1 and ICAM-1 on Endothelial Cells

Antibody coated microparticles were used to characterize the role PKCδ plays in upregulation of adhesion molecules on endothelial cells post-IR (Prabhakarpandian et al., 2011, Microcirculation, 18:380-389; Lamberti et al., 2013, Microvasc Res, 89:107-114). Adhesion of microparticles coated with antibodies to E-selectin, P-selectin, ICAM-1, or VCAM-1 to 2 Gy irradiated endothelial cells was measured 48 hours post-IR under the experimental conditions described earlier. Irradiation at 2Gy significantly increased the adhesion of anti-ICAM-1 and anti-VCAM-1, but not anti-E-selectin or anti-P-selectin, coated microparticles to endothelial cells (FIG. 8). VCAM-1 demonstrated the largest increase in expression. PKCδ inhibition significantly reduced adhesion of anti-P-selectin, anti-VCAM-1, and anti-ICAM-1, but not anti-E-selectin, coated microparticles to irradiated endothelial cells (FIG. 8). Hence, PKCδ regulates IR-induced ICAM-1, VCAM-1, and P-selectin expression in endothelial cells.

### Role of PKCδ and Requirement for Tyrosine Phosphorylation in Regulating Radiation-Induced Expression of Selectins and Adhesion Molecules Required for Neutrophil-Microvascular Endothelial Cell Interaction and Neutrophil Migration

Using human and mouse pulmonary microvascular endothelial cells, the effect of radiation on PKCδ activation and tyrosine phosphorylation is determined. Nothing is known about the regulation of PKCδ in response to radiation in PKCδY155F KI and PKCδY311F KI mice and whether PKCδ Tyr155 or Tyr 311 phosphorylation is required for other tyrosine phosphorylation steps and for expression of selectins and adhesion molecules critical for neutrophil adherence and transmigration. On the endothelium, selectins (P-selectin & E-selectin), PSGL-1, adhesion molecules (ICAM-1, ICAM-2, VCAM-1, PECAM-1, JAM-C, VE-cadherin) are critical regulators of neutrophil rolling, adherence, and transmigration (Chavakis et al., 2004, Journal of Biological Chemistry, 279(53):55602-8; Reutershan and Ley, 2004, Crit Care, 8(6):453-61; Reutershan et al., 2007, Am J Respir Crit Care Med, 175(10):1027-35; Doerschuk et al., 2000, American Journal of Respiratory Cell and Molecular Biology, 23(2):133-6). In vitro, PKCδ inhibition decreased adhesion molecule expression (Mondrinos et al., 2014, The American Journal of Pathology, 184(1):200-213; Soroush et al., 2016, Journal of Leukocyte Biology, 100(5):1027-1035). Adhesion molecule expression may be regulated by PKCδ through activation of MAPK (p38, JNK and ERK) and the transcription factors NF-κB and AP-1 (Kilpatrick et al., 2002, Am J Physiol Cell Physiol, 283(1):C48-57; Kilpatrick et al., 2004, Am J Physiol Cell Physiol, 287(3):C633-42; Mondrinos et al., 2014, The American Journal of Pathology, 184(1):200-13; Rahman et al., 2001, Mol Cell Biol, 21(16):5554-65; Ramnath et al., 2008, Am J Physiol Cell Physiol, 294(3):C683-92). Pharmacologic tools (PKCδ TAT inhibitor) as described previously (Mondrinos et al., 2014, The American Journal of Pathology, 184(1):200-13) and genetic tools in endothelial cells isolated from WT, PKCδ-/-, PKCδY311F and PKCδY155F mice are employed to determine the role of PKCδ in critical signaling pathways which regulate expression of key molecules involved in neutrophil recruitment post-IR. These studies provide important mechanistic information about neutrophil-endothelial cell interactions and transmigration for in vivo studies.

Human primary pulmonary microvascular endothelial cells are purchased from ScienCell (Carlsbad, Calif.). Murine primary pulmonary endothelial cells are isolated from WT, PKCδY155F KI and PKCδY311F KI mice (Bein et al., 2012, J Trauma Acute Care Surg, 73(6):1450-1456). Human endothelial cells are cultured using endothelial growth medium 2 (Lonza) with added bovine brain extract, vascular endothelial growth factor, epidermal growth factor, gentamicin, and hydrocortisone according to the manufacturer's specifications as described previously (Mondrinos et al., 2014, The American Journal of Pathology, 184(1):200-13).

Cells are obtained at passage 3 and are used up to passage 6. Using established protocols (Prabhakarpandian et al., 2001, Microcirc, 8:355-364; Kiani et al., 2002, Pharm Res, 19(9):1317-1322), endothelial cells are irradiated with X-ray (4, 8, or 12 Gy at 0.75 Gy/min, filter: Al 0.5 mm+Cu 0.5 mm, 320 kVp unit) (5 samples/group). C57BL/6 mice have LD50/30 (50% mortality at 30 days) to LD70/30 with a radiation dose of 7.7-8.0 Gy (Chua et al., 2012, Health Phys, 103(4):356-366). Endothelial cells in vitro may be more sensitive to high dose radiation and the range of radiation doses is adjusted if necessary. Sham irradiated cells are used as a negative control and TNF (10 U/ml) treated cells as a positive control. It has been shown that vascular adhesion molecules are upregulated similarly in response to X-ray and gamma-ray irradiation (Prabhakarpandian et al., 2001, Microcirc, 8:355-364).

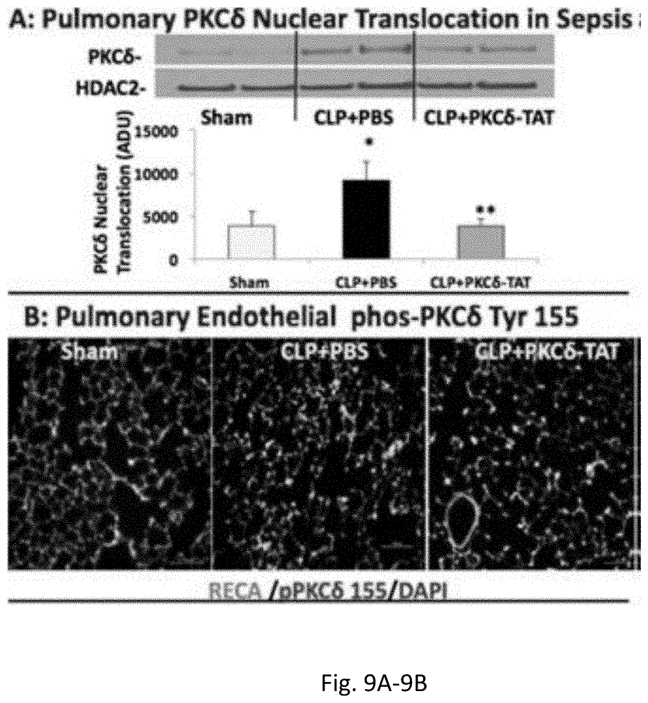

PKCδ expression and activation are determined at 0, 1, 8, 24 and 48 hours post-IR. PKCδ expression is determined by western blotting with a pan-PKCδ antibody (Cell Signaling Technology). Equal protein loading is assessed by reprobing for β-actin and PKCδ expression is normalized β-actin. PKCδ tyrosine phosphorylation is determined by Western blotting using phospho-PKCδ antibodies against tyrosine residues located in the regulatory (Tyr 52, 64, 155, 187), hinge region (Tyr 311, 332) and catalytic domain (Tyr 525, 565) of PKCδ (Cell Signaling Technology, Antibodies-online.com, Santa Cruz Biotechnology) (FIG. 9) (Mondrinos et al., 2015, Journal of Pharmacology and Experimental Therapeutics, 355(1):86-98).

To examine the roles of PKCδ in regulating selectin and adhesion molecules, human microvascular and murine pulmonary microvascular endothelial cells isolated from WT, PKCδ-/-, PKCδY155F KI and PKCδY311F KI mice are cultured, and irradiated with X-ray (4, 8, or 12 Gy at 0.75 Gy/min, 320 kVp unit) (5 samples/group) as described above. At 24 and 48 hours post-IR, cell supernatants are collected for cytokine/chemokine profile analysis. Expression of ICAM-1, ICAM-2, PECAM-1, VCAM-1 and JAM-C are determined by ELISA at 24 and 48 hours post-radiation as described previously (Mondrinos et al., 2014, The American Journal of Pathology, 184(1):200-13). P-selectin and Eselectin, PSGL-1 and VE-cadherin expression are determined by flow cytometry with murine and human antibodies and isotype matched controls (BD Biosciences). The effect of PKCδ inhibition and the role of PKCδ tyrosine phosphorylation are determined to ascertain whether δ-PKC controls a specific cytokine/chemokine expression pattern or has a more global effect. KC and MIP-2 are important regulators of murine neutrophil recruitment in response to systemic inflammation (Chen et al., 2001, Proc Natl Acad Sci USA, 98(20):11114-11119). KC (R & D Systems) and MIP-2 (BioSource) levels are measured by ELISA (Kilpatrick et al., 2011, Journal of Leukocyte Biology, 89(1):3-10). In human PMVEC, CXCL8, CXCL5 and CXCL1 are important chemoattractants produced by endothelial cells in response to inflammation are determined by ELISA (R & D Systems). To determine if δ-PKC inhibition reduces inflammatory cytokine expression and increases anti-inflammatory cytokine expression, the cytokines, IL1A, IL1B, IL2, IL4, IL6, IL10, IL12, IL13, IFNγ, TNFα, GM-CSF, and RANTES are determined in endothelial cell supernatants by the Mouse or Human Inflammatory Cytokines Multi-Analyte ELISArray Kit (Qiagen). These studies show PKCδ inhibition promotes an anti-inflammatory milieu. Activation of the transcription factors NFκB and AP-1 activity are determined by nuclear translocation and EMSA as described previously (Kilpatrick et al., 2002, Am J Physiol Cell Physiol, 283(1):C48-57; Mondrinos et al., 2014, The American Journal of Pathology, 184(1):200-13). MAPK (ERK, p38 and JNK) activation is monitored in cell lysates by immunoblotting with phospho-antibodies for ERK1/2 (Thr202/Tyr204), JNK (Thr183/Tyr185) and p38 MAPK (Thr180/Tyr182) as described previously (Kilpatrick et al., 2006, J Leuk Biol, 80:1512-1521). Equal loading of MAPKs is confirmed by reprobing with antibodies that recognize phosphorylated and non-phosphorylated forms of the specific MAPK.

### Determine the Role of PKCδ and PKCδ Specific Tyrosine Phosphorylation Sites in Regulating Radiation-Induced Endothelial Cell Apoptosis

Human microvascular endothelial cells and murine microvascular endothelial cells isolated from WT, PKCδ-/-, PKCδY155F KI and PKCδY311F KI mice are cultured and irradiated with X-ray (4, 8, or 12 Gy at 0.75 Gy/min, 320 kVp unit) (5 samples/group). Following irradiation, PKCδ nuclear translocation, PKCδ cleavage, caspase 3 activity and endothelial cell apoptosis (TUNEL) are determined at 0, 8, 24 and 48 hours post-IR. PKCδ translocation are determined by preparing membrane, cytosolic and nuclear fractions from cell lysates and PKCδ translocation determined by Western blotting using a pan-PKCδ antibody (Cell Signaling Technology) as described previously (Mondrinos et al., 2014, The American Journal of Pathology, 184(1):200-13; Mondrinos et al., 2015, Journal of Pharmacology and Experimental Therapeutics, 355(1):86-98; Korchak et al., 2007, Biochim Biophys Acta, 1773(3):440-449). Purity of nuclear fractions are determined by probing fractions for cytoplasmic (lactate dehydrogenase) and nuclear (HDAC-2) markers. PKCδ cleavage is determined in cell lysates through western blotting for full-length and cleaved PKCδ using an antibody directed against the C terminus of PKCδ (sc-8402; Santa Cruz Biotechnology) as described previously (Mondrinos et al., 2015, Journal of Pharmacology and Experimental Therapeutics, 355(1):86-98). Endothelial cell apoptosis is determined by TUNEL assay for DNA fragmentation at 0, 8, 24 and 48 hours post-radiation (Kilpatrick et al., 2006, J Leuk Biol, 80:1512-1521). Caspase 3 activity is determined by monitoring cleavage of fluorochrome-conjugated peptide substrate (caspase 3 (Z-DEVD-R110)) as described previously (Kilpatrick et al., 2006, J Leuk Biol, 80:1512-1521).

### Determine the Role of Endothelial Cell Heterogeneity in PKCδ Phosphorylation and Activation in Response to Radiation Exposure

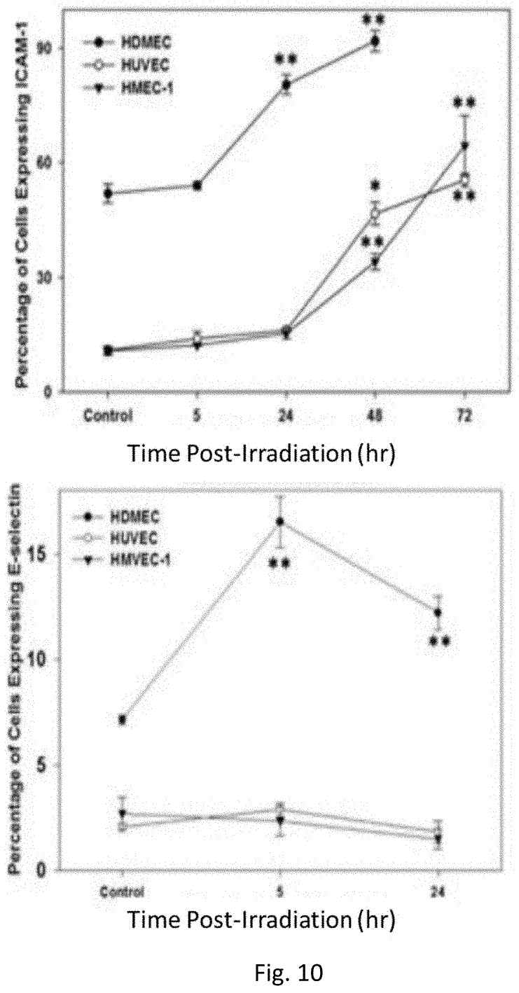

Microvascular endothelial cells are acutely sensitive to radiation and radiation-induced alterations in endothelial structure and function is a critical factor in organ damage through endothelial cell activation, increased leukocyte-endothelial cell interactions, and increased permeability and apoptosis (Molema, 2010, Endothelial Dysfunction and Inflammation. Basel: Springer Basel, p. 15-35; Prabhakarpandian et al., 2001, Microcirc, 8:355-364; Rossaint and Zarbock, 2013, Journal of Innate Immunity, 5(4):348-357). Endothelial cell heterogeneity in different organs such as intestine, lung, and brain are well described (Nolan Daniel et al., 2013, Developmental Cell, 26(2):204-219) and organ-specific variations in endothelial structure, function and microenvironments lead to differential responses to IR. It has been shown that while ICAM-1 is upregulated on irradiated human dermal microvascular endothelial cells (HDMEC), HUVEC, and transformed human microvascular endothelial cells (HMEC-1), E-selectin is only upregulated on irradiated HDMEC (FIG. 10) (Prabhakarpandian et al., 2001, Microcirc, 8:355-364). PKCδ is an important regulator of adhesion molecule expression and differences in expression or activation patterns may result in altered endothelial cell responses to radiation. In support of this concept, heterogeneity in apoptotic responses of lung endothelial cells compared to heart endothelial cells to oxidative stress was observed (Grinnell et al., 2012, Journal of Cellular Physiology, 227(5):1899-910). The increased apoptosis in lung endothelium was associated with increased PKCδ expression and PKCδ cleavage products indicating differential expression of PKCδ may be a critical factor in endothelial responsiveness. Endothelial cells from different organs are tested to determine if they display distinct differences in response to radiation through differential activation of adhesion molecule expression, cytokine/chemokine profiles and/or the apoptotic program. Further the hypothesis that differential endothelial cell responsiveness to radiation is the result of altered PKCδ expression, tyrosine phosphorylation patterns, nuclear translocation and enzyme cleavage is tested.

Murine intestinal, pulmonary and brain microvascular endothelial cells are isolated from WT, PKCδ-/-, PKCδY155F KI and PKCδY311F KI mice. These cells are irradiated as described above. At 0, 1, 8, 24 and 48 hours post-IR, cell lysates are prepared and PKCδ expression, tyrosine phosphorylation are determined. The impact of endothelial cell heterogeneity on radiation-induced activation of apoptotic programs is ascertained. At 0, 8, 24 and 48 hours post-IR cell lysates are prepared and PKCδ nuclear translocation, PKCδ cleavage, caspase 3 activity and cellular apoptosis are determined. Endothelial cells from PKCδ-/- are not used for studies analyzing PKCδ activation, phosphorylation and cleavage, but are used in studies examining caspase 3 activation and apoptosis. Next, the impact of endothelial cell heterogeneity on cell activation in response to radiation is determined. Under the same experimental conditions as described above, at 24 and 48 hours post-IR, cell supernatants are collected and cytokine/chemokine profiles are determined. Cell signaling is determined by measuring activation of MAP kinases and transcription factors and endothelial selectins/adhesion molecule expression is determined. In a second series of experiments, human intestinal microvascular cells, pulmonary microvascular endothelial cells and brain microvascular endothelial cells are obtained from ScienCell (Carlsbad, Calif.). To test therapeutic relevancy, human endothelial cells are exposed to radiation as described above and treated with a) the PKCδ-TAT peptide (2 μM) orb) vehicle (PBS) 24 hours after radiation treatment. Endothelial cell activation is determined 48 hours post radiation (24 hours post PKCδ inhibition) by determining selectin/adhesion molecule expression and development of apoptosis.

Without being bound by a particular theory, it is expected that radiation activates PKCδ in microvascular endothelial cells. PKCδ is activated in response to radiation in other cell types (Wie et al., 2014, Journal of Biological Chemistry, 289(15):10900-10908; Mitsutake et al., 2001, Oncogene, 20(8):989-996; Reyland and Jones, 2016, Pharmacology & Therapeutics, 165:1-13) and inhibiting PKCδ activity in salivary glands was protective against radiation damage (Wie et al., 2014, Journal of Biological Chemistry, 289(15):10900-10908). Further, radiation exposure triggers discrete tyrosine phosphorylation patterns including Tyr 64, 155 and 311, phosphorylation sites critical for PKCδ nuclear translocation and cleavage, alterations required for the induction of apoptosis (Humphries et al., 2007, Oncogene, 27(21):3045-53; Zhao et al., 2012, Archivum Immunologiae et Therapiae Experimentalis, 60(5):361-372; Pabla et al., 2011, The Journal of Clinical Investigation, 121(7):2709-22). PKCδTyr311 and Tyr155 phosphorylation in endothelial cells is required to initiate apoptosis in response to radiation. Endothelial cells from PKCδY155F KI and PKCδY311F KI mice, similar to PKCδ-/- KO, are protected from inflammatory responses to radiation exposure, attenuated proinflammatory signaling, reduced selectin and adhesion molecule expression and decreased apoptosis. In vivo studies (FIG. 9) indicate that PKCδTyr155 phosphorylation is critical for neutrophil migration. These studies provide important information about the role of endothelial PKCδ in regulating expression of key molecules involved in the neutrophil adhesion cascade. Based on the data presented herein which showed PKCδ inhibition had a greater impact on neutrophil migration rather than adherence, it was expected that PKCδ would be a key regulator of adhesion molecules critical for neutrophil migration such as ICAMs and VCAM-1 (Kolaczkowska and Kubes, 2013, Nat Rev Immunol, 13(3):159-175). The ability to use pharmacologic and genetic methods to selectively inhibit PKCδ provided a powerful tool to dissect signaling pathways. Previously a role for PKCδ in regulating adhesion molecules expression in response to systemic inflammation (sepsis in vivo and TNF/IL-1 in vitro) was demonstrated (Mondrinos et al., 2014, The American Journal of Pathology, 184(1):200-13; Soroush et al., 2016, Journal of Leukocyte Biology, 100(5):1027-35; Mondrinos et al., 2015, Journal of Pharmacology and Experimental Therapeutics, 355(1):86-98). Thus, PKCδ is an important regulator in response to radiation exposure. Cells from PKCδ-/- KO, PKCδY155F and PKCδY311F KI mice provide important mechanistic information about PKCδ regulation of the MAPKs and the transcription factors NFκB and AP-1. Tyr155 phosphorylation regulates PKCδ nuclear translocation which is required for JNK activation but not p38 MAPK (Gomel et al., 2007, Molecular Cancer Research, 5(6):627-639). ERK activation is independent of Tyr155 in glioma cells (Sims et al., 2010, Nat Rev Immunol, 10(2):89-102). Thus, blocking Tyr155 phosphorylation attenuates JNK activation but not ERK or p38 MAPK. Furthermore, PKCδ binds NF-κB RelA subunit in the nucleus so inhibition of PKCδ nuclear translocation should prevent NκB activation. Interpretation of in vivo results obtained by intravital microscopy and in vitro in the microfluidics assay are used to predict specific steps in the cascade regulated by PKCδ in irradiated cells. Finally, it is anticipated that endothelial cells which are more sensitive to radiation (i.e. intestinal microvascular endothelial cells) have increased PKCδ expression and increased radiation-induced nuclear translocation, kinase cleavage and apoptosis as compared to endothelial cells which are more resistant to radiation-induced cellular damage (i.e. lung and brain microvascular cells).

### Example 2: PKCδ is a Critical Regulator of Leukocyte Migration Through Vascular Endothelium After Radiation Injury

Neutrophil recruitment to irradiated endothelium is a multi-step cascade that is a dynamic phenomenon and its understanding requires real-time monitoring of the entire process. The crosstalk between neutrophils and endothelial cells is composed of a series of interactions which orchestrate rolling, adhesion and transmigration (Maniatis and Orfanos, 2008, Current Opinion in Critical Care, 14(1):22-30; Reutershan and Ley, 2004, Crit Care, 8(6):453-461; Orfanos et al., 2004, Intensive Care Medicine, 30(9):1702-1714). Ultimately, arrested neutrophils migrate across endothelial cells to inflamed tissues via a multi-step process controlled by concurrent chemoattractant-dependent signals, adhesive events and hemodynamic shear forces (Ley et al., 2007, Nat Rev Immunol, 7(9):678-689; Kolaczkowska and Kubes, 2013, Nat Rev Immunol, 13(3):159-175; Phillipson and Kubes, 2011, Nat Med, 17(11):1381-1390; Molteni et al., 2006, Current Opinion in Cell Biology, 18(5):491-498). The data presented in FIG. 5 through FIG. 9 indicate a role for PKCδ in regulating neutrophil migration and loss of barrier function after irradiation, but do not address specific mechanisms or identify specific steps.

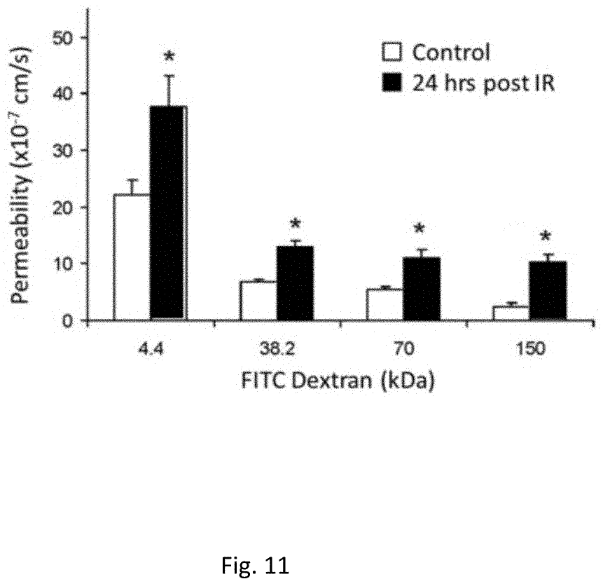

Intravital microscopy provides a powerful technique to study the micro-circulation and permits continuous, direct, real-time measurement of multiple parameters including flow, leukocyte-endothelial interaction and permeability. Intravital microscopy has been used to study mechanisms and clinical manifestations of radiation injury and shown increased permeability of cremaster muscle microvasculature to FITC-dextran 4.4-150 kDa after 20 Gy local irradiation (FIG. 11) (Yuan et al., 2003, Brain Res, 969(1-2):59-69). Changes in microvascular structure, function, and tissue oxygenation have been studied up to 180 days post-IR (Yuan et al., 2003, Brain Res, 969(1-2):59-69; Yuan et al., 2006, Int J Radiat Oncol Biol Phys, 66(3):860-866; Yuan et al., 2005, Radiat Res, 163(5):544-551; Kiani et al., 2002, Pharm Res, 19(9):1317-1322; Ansari et al., 2007, Radiat Res, 167(1):80-86; Roth et al., 1999, Radiat Res, 151(3):270-277; Gaber et al., 2004, Brain ResProtoc, 13(1):1-10; Nguyen et al., 2000, Radiat Res, 154:531-536; Kiani et al., 2003, Adv Exp Med Biol, 510:391-395; Kiani et al., 2003, J Radiat Res, 44(1):15-21).

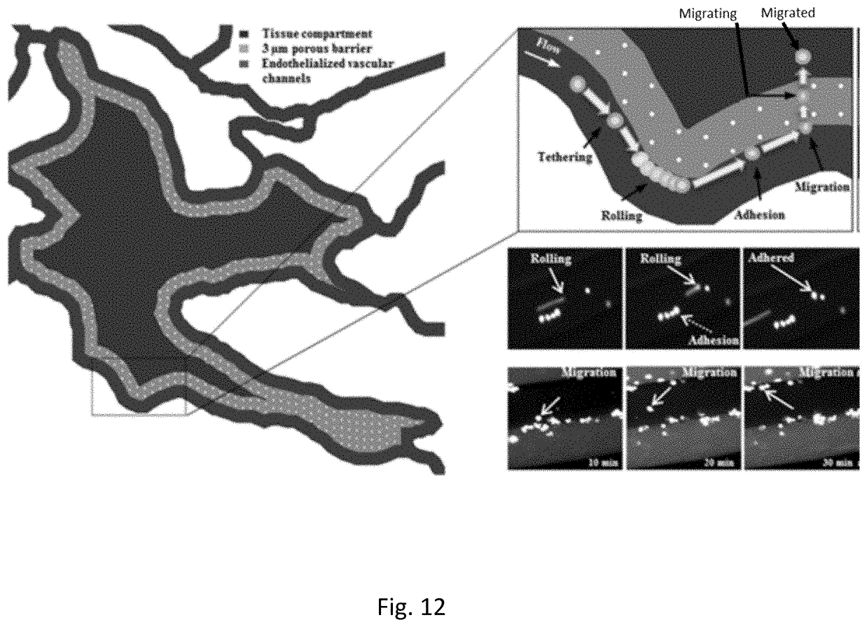

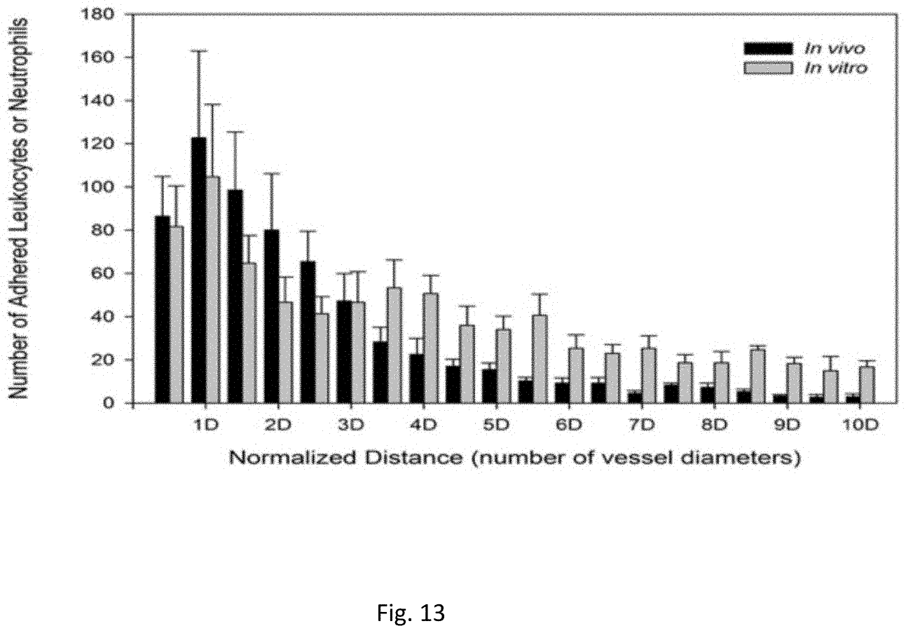

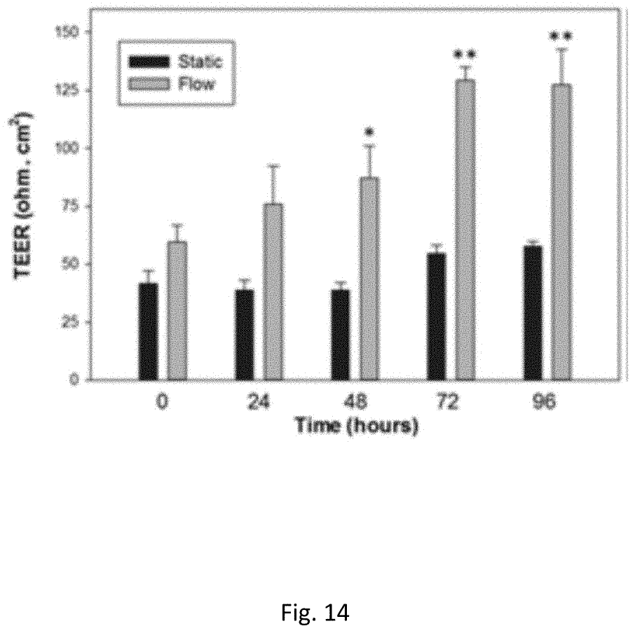

An integrated microfluidic assay is employed for studying the neutrophil adhesion cascade in a physiologically realistic environment, encompassing circulation, rolling, adhesion and migration of neutrophils (FIG. 12) (Rosano et al., 2009, Biomed Microdevices, 11:1051-1057; Soroush et al., 2016, Journal of Leukocyte Biology, 100(5):1027-35; Lamberti et al., 2014, AnalChem, 86(16):8344-8351). This novel in vitro model recreates physiologically realistic (topology and shear conditions) micro-vascular environments that can be used to study the complete adhesion cascade. The biomimetic microfluidic assay (bMFA) has a vascular compartment, reproduced from in vivo images, in which endothelial cells form a complete lumen and a tissue compartment which can be filled with chemoattractants (e.g. fMLP); these two compartments are connected by 3 μm wide gaps, an optimum size for neutrophil migration (Yona et al., 2010, Curr Protoc Immunol, Chapter 14:Unit 14 5; Entschladen et al., 2005, Experimental Cell Research, 307(2):418-26; Chen et al., 2005, Methods Mol Biol, 294:15-22). Neutrophil rolling and adhesion patterns in bMFA are similar to those observed in vivo by intravital microscopy (Prabhakarpandian et al., 2011, Microvasc Res, 82:210-220; Tousi et al., 2010, Microvasc Res, 80(3):384-388; Lamberti et al., 2014, AnalChem, 86(16):8344-8351). Both in vivo and in vitro, neutrophils preferentially adhere to activated endothelial cells near bifurcations with rolling and spatial adhesion patterns in close agreement (FIG. 13) (Lamberti et al., 2014, AnalChem, 86(16):8344-8351). Neutrophil adhesion was minimal in high shear regions (shear rate >120 1/sec) and maximal in low shear regions, indicating fluidic shear strongly influences cell adhesion in these microvascular networks (Lamberti et al., 2014, AnalChem, 86(16):8344-8351). The bMFA has electrodes in two compartments for transendothelial electrical resistance (TEER) measurements (Deosarkar et al., 2015, PLoS One, 10(11):e0142725). Under static conditions, TEER of HUVEC cells remained relatively constant over 96 hours at ˜50 ohm.cm² (FIG. 14). Under shear flow, tight junctional endothelial integrity was significantly enhanced and TEER increased by 2.5 fold. Thus, in vivo (intravital microscopy) and in vitro (bMFA) tools are used to evaluate a novel therapeutic paradigm which targets PKCδ and neutrophil-endothelial interactions to protect vascular endothelium and attenuate radiation-induced tissue damage.

The effect of global deletion PKCδ and the roles of PKCδ Tyr155 and Tyr 311 phosphorylation on neutrophil migration is determined in vivo and in vitro. Pharmacologic studies using a PKCδ inhibitor are used to examine the therapeutic effects of PKCδ-TAT inhibitor administration after irradiation and rule out other compensatory effects that may be associated with gene deletion in mice. Through the use of genetic and pharmacologic tools, the role of PKCδ in neutrophil-endothelial cell interaction and migration after irradiation is ascertained. Studies using intravital microscopy in vivo in conjunction with bMFA permit the analysis of the role of PKCδ in regulating individual steps in neutrophil migration through irradiated vasculature.

### The Role of PKCδ on Neutrophil Rolling/Adhesion and Migration In Vitro Using a Microfluidics Assay

The effect of PKCδ inhibition (genetic and pharmacologic) on spatial distribution of adhering/migrating leukocytes are determined using bMFA. Endothelial cells from: 1) WT, 2) PKCδ-/-, 3) PKCδY155F, 4) PKCδY311F mice are prepared and the role of PKCδ in regulating human neutrophil-endothelial cell interaction is measured to determine the degree of correspondence between human vs. mouse endothelial cells (Perlman et al., 2013, American Journal of Respiratory and Critical Care Medicine, 187(9):898-900; Seok et al., 2013, Proceedings of the National Academy of Sciences, 110(9):3507-3512; Osuchowski et al., 2014, Shock, 41(6):463-475). Endothelial cells from different organs have differential responses to radiation damage (Groans et al., 2012, Medical Consequences of Radiological and Nuclear Weapons. Fort Detrick, Md.: Borden Institute; p. 17-38). Thus, human and mouse intestinal, lung, and brain endothelial cells are studied from organs with varying degrees of radiation sensitivity. Human neutrophils are isolated as described (Soroush et al., 2016, Journal of Leukocyte Biology, 100(5):1027-35; Lamberti et al., 2014, AnalChem, 86(16):8344-8351). Mouse bone marrow cells are flushed from femurs and tibias and neutrophils obtained from a discontinuous Percoll gradient. RBCs are removed by hypotonic lysis and neutrophil purity determined by Gr-1 staining (Alves-Filho et al., 2009, Proceedings of the National Academy of Sciences, 106(10):4018-4023). Neutrophils are labeled using CFDA SE probe (Invitrogen, Eugene, Oreg.).

Using an established protocol (Rosano et al., 2009, Biomed Microdevices, 11:1051-1057; Lamberti et al., 2014, AnalChem, 86(16):8344-8351; Lamberti et al., 2013, Microvasc Res, 89:107-114), a confluent tubular monolayer of endothelial cells is established under shear flow (inlet flow rate of 1μl/min). Confluent endothelial cells are irradiated with X-ray (4, 8 and 12 Gy) (5 bMFA/group). Sham irradiated assays are used as negative control and TNFα (10U/ml, 24 hours) as a positive control. Murine neutrophils (105 cells/ml ) are introduced in the vascular compartment 48 hours post-IR or TNF exposure. Cell rolling, adhesion and migration into the tissue compartment is captured in real-time by scanning the entire network continuously for 2 hours using automated tracking (Soroush et al., 2016, Journal of Leukocyte Biology, 100(5):1027-35; Lamberti et al., 2014, AnalChem, 86(16):8344-8351) (FIG. 11). It should be noted that even though the shear rate at the inlet is fixed, the shear rate in the network varies (15-500 sec−1) at different locations, and cannot be calculated by a simple mathematical formula. A CFD (computational fluid dynamics)-based model has been developed to calculate various flow parameters (e.g. shear stress) at different locations in the network (Prabhakarpandian et al., Biomedical Microdevices, 10(4):585-595). The number of rolling cells is quantified at each location. Cells that don't move for 30 sec are considered adherent. Plots of shear rate vs. number of rolling cells and shear rate vs. number of adhered cells at different locations are generated. A notable feature of the bMFA is the ability to resolve the effect of shear rate on adhesion in bifurcations vs. linear sections of the network. In a series of translational experiments, human microvascular endothelial cells are irradiated as described above and 24 hours post-IR, cells are treated with the PKCδ inhibitor (2 μM) under shear flow as described (Kilpatrick et al., 2004, Am J Physiol Cell Physiol, 287(3):C633-42; Kilpatrick et al., 2006, J Leuk Biol, 80:1512-1521; Kilpatrick et al., 2010, J Leukoc Biol, 87(1):153-164; Soroush et al., 2016, Journal of Leukocyte Biology, 100(5):1027-1035). Human neutrophils (10⁵ cells/ml) are introduced in the vascular compartment 48 hours post-IR (24 hours post PKCδ inhibitor treatment) to study the complete cascade.

### In Vivo Analysis of the Impact of Ionizing Radiation on Leukocyte Rolling, Adhesion and Migration in the Microvascular Networks of a Mouse Cremaster Model

Intravital microscopy is employed for real-time assessment of neutrophil trafficking to examine the effect of PKCδ inhibition by genetic (PKCδ-/-, PKCδY155F KI and PKCδY311F KI) or pharmacologic inhibition (PKCδ TAT inhibitor) in regulating neutrophil rolling, adhesion and migration. For mechanistic studies, 4 groups are used: 1) WT, 2) PKCδ-/-, 3) PKCδY155F KI, and 4) PKCδY311F KI mice. Following established protocols (Kiani et al., 2002, Pharm Res, 19(9):1317-1322), the mice are irradiated (IR) with a whole body dose of X-ray (4, 8, or 12 Gy, filter: Al 0.5 mm+Cu 0.5 mm, 0.75 Gy/min, 320 kVp unit) (6 mice/group). Radiation doses are verified to <5% total uncertainties using clinical machine output check, independent dose/monitor unit calculation, and dose measurements. For translational studies, WT mice are irradiated and then treated 24 hours post-IR subcutaneously (SC) with the PKCδ inhibitor (0.2 mg /kg) (Qi et al., 2008, J Clin Invest, 118(1):173-182) or PBS vehicle. These doses are selected to cover a range of radiation exposures since C57BL/6 mice have LD50/30 (50% mortality at 30 days) to LD70/30 radiation dose of 7.7-8.0 Gy (Chua et al., 2012, Health Phys, 103(4):356-366). Sham irradiated animals are used as control. At 48 hours, 1 week, or 1 month post-IR, animals are anesthetized, intubated, catheterized (left femoral vein), and placed on a surgical board where the right cremaster muscle is pinned to a flat sheet with minimal disruption to the tissue. Rhodamine 6G (0.4 mg/kg) is injected via the tail vein to fluorescently label the leukocytes. Images are acquired continuously for 1 hour to quantify neutrophil rolling, adhesion and migration in the bifurcations and linear sections of the vessels. Male mice are used for these studies as the cremaster muscle is only present in male rodents. Radiation sensitivity between sexes in C57BL mice has been reported to not be significantly different (Abrams, 1951, Proc Soc Exp Biol Med, 76(4):729-732; Kallman and Kohn, 1956, Radiat Res, 5(4):309-317; Plett et al., 2012, Health Phys, 103(4):343-355).

Global deletion of PKCδ or altered PKCδ Tyr155 or Tyr 311 phosphorylation can have a significant impact on in vitro and in vivo neutrophil migration across irradiated endothelial cells. The intravital microscopy approach in conjunction with bMFA provides unique insight into neutrophil-endothelial interactions. With these techniques and KO and KI mice specific steps in rolling, adhesion and migration are identified as being regulated by PKCδ and specific tyrosine phosphorylation sites. In vitro preliminary studies identified endothelial PKCδ a critical regulator of cell adhesion, migration and permeability in irradiated endothelial cells (FIG. 3-6). Also, PKCδ-/- mice demonstrated a protection from radiation-induced damage (Humphries et al., 2006, Journal of Biological Chemistry, 281(14):9728-9737; Reyland and Jones, 2016, Pharmacology & Therapeutics, 165:1-13). Thus, it is consistent that PKCδ-/-, PKCδY155F, and PKCδ-TAT peptide inhibitor treated cells have decreased neutrophil migration in response to radiation exposure.

Mice are monitored for 1 month post-IR for early effects of radiation exposure. Late effects of radiation exposure can be observed months to years post-IR, but without being bound by a particular theory, it is believed that by preventing the early inflammatory response post-IR, many of the late effects can be prevented (Williams and McBride, 2011, Int J Radiat Biol, 87(8):851-868; Korpela and Liu, 2014, Radiat Oncol, 9:266; Paris et al., 2001, Science, 293(5528):293-297).

### Example 3: PKCδ Regulates Vascular Permeability and Endothelial Cell Damage After Radiation Injury

While critical to host defense, neutrophils can also damage endothelial cells characterized by increased intracellular gaps, apoptosis, sloughing of endothelial cells and increased vascular permeability (Prown et al., 2006, The Lancet, 368(9530):157-169; Harbour et al., 1991, Circ Shock, 35(3):181-191). Radiation exposure induces organ damage (Panes and Granger, 1996, Gastroenterology, 111(4):981-989; Moeller et al., 2004, Cancer Cell, 5(5):429-441; Hauer-Jensen et al., 2004, Crit Care Med, 32(5 Suppl):5325-5330) and in vitro radiation-induced endothelial damage, including leukocyte adhesion, migration, and increased permeability are attenuated by the PKCδ inhibitor (FIG. 4 through FIG. 8). It is not known if the endothelial protective effects of the PKCδ inhibitor have organ protective effects in vivo. The mechanisms by which PKCδ regulates radiation-mediated endothelial cell damage and vascular permeability are not known and are investigated using intravital microscopy and the in vitro biomimetic microfluidic assay.

### In Vitro Analysis of Vascular Endothelial Permeability and Endothelial Cell Damage

The bMFA is used to determine the effect of PKCδ inhibition (genetic and pharmacologic) on endothelial cell damage and permeability in response to radiation injury. Endothelial cells isolated from 1) WT, 2) PKCδ-/-, 3) PKCδY155F, and 4) PKCδY311F mice are used. In parallel studies human microvascular endothelial cells re treated with the PKCδ inhibitor (2 μM) 24 hours post-IR.

The role of PKCδ in radiation-induced endothelial permeability is determined by monitoring TEER changes across endothelial cells in the bMFA (Deosarkar et al., 2015, PLoS One, 10(11):e0142725). TEER is measured using a Zurich Instruments HF2IS Impedance Spectroscope. Silver chloride electrodes are placed on either side of the endothelial cells in the vascular and tissue compartments. Impedance measurements are acquired at 10 kHz with a voltage of 10 mV (FIG. 14). Baseline TEER of the confluent endothelial cell monolayer is determined and then at 48 hours post-IR under the experimental conditions described in Example 2. Vascular endothelial permeability is measured under the same condition by FITC-albumin migration across endothelial cells (Deosarkar et al., 2015, PLoS One, 10(11):e0142725). Using the methodology employed in FIG. 5, FITC-albumin is added to the vascular compartment of the bMFA and fluorescence levels are measured in both vascular and tissue compartments every 5 minutes for 2 hours and permeability is determined using standard curves for FITC albumin.

Endothelial cell damage is monitored by endothelial thrombomodulin levels and its release into the perfusate. Thrombomodulin levels are measured with mouse or human thrombomodulin /BDCA-3 DuoSet ELISA kits (R & D Systems, MN) (Martin et al., 2014, PloS one, 9(9):e108254). Both endothelial cells and perfusate are collected and used to monitor thrombomodulin content and release. Tissue associated thrombomodulin is measured in cell lysates. Neutrophils are flushed from the vascular compartment, endothelial cells harvested with dissociation media and cell homogenates prepared. Endothelial cell damage is measured by annexin V expression. Neutrophils are flushed from the vascular compartment, endothelial cells harvested, and incubated with PE-anti-CD146 (a marker of endothelial cells), allophycocyanin-Annexin V and PE/Cy5-anti-CD45 (a marker of leukocytes) (BD Pharmingen). The % of cell cytotoxicity is determined by cells double-positive for CD146 and Annexin V in the CD45-negative gate by flow cytometry (Villanueva et al., 2011, J Immunol, 187(1):538-552).

### In Vivo Analysis of the Impact of Radiation on Endothelial Permeability

Intravital microscopy is used to examine the effect of global genetic modification of PKCδ (PKCδ-/- , PKCδ Y155F KI and PKCδ Y311F KI) in regulating endothelial permeability in the microvascular networks of a mouse cremaster model.

Six animal groups are used: 1) WT, 2) PKCδ-/- , 3) PKCδY155F KI, 4) PKCδY311F KI, 5) WT+SC PKCδ TAT inhibitor (0.2 mg/kg) administered 24 hours post-IR and 6) W +SC PBS vehicle (24 hours post-IR). The effect of X-ray (2, 4, 8 Gy) on vascular permeability is evaluated by intravital microscopy at 48 hours, 1 week, and 1 month post-IR. Vascular permeability is determined by injection of FITC-albumin (50 mg/kg). Permeability is calculated from ratio of fluorescence intensity in the venules to the perivenular interstitium following albumin injection measured at 5 minutes intervals for 2 hours (Yuan et al., 2003, Brain Res, 969(1-2):59-69; Gaber et al., 2004, Brain Res Protoc, 13(1):1-10; Schmidt et al., 1997, Critical Care Medicine, 25(5):858-863; Stagg et al., 2013, J Trauma Acute Care Surg, 75(6):1040-1046; El-Sayed et al., 2001, Pharmaceutical Research, 18(1):23-28). In a second series of experiments, the in vivo therapeutic efficacy of pharmacological inhibition of PKCδ for treating radiation injury is evaluated. Intravital microscopy is used to determine if subcutaneous administration of PKCδ TAT peptide inhibitor (0.2 ug/kg) can reduce endothelial permeability in the microvascular networks of a mouse cremaster model. Experimental conditions are as described in Example 2.

### Role of PKCδ in Regulating Neutrophil Influx, Organ Damage, and Survival

After irradiation, inflammatory mediators damage vascular endothelium resulting in increased permeability and neutrophil migration which is associated with tissue damage, organ failure, and increased mortality (Panes and Granger, 1996, Gastroenterology, 111(4):981-989; Moeller et al., 2004, Cancer Cell, 5(5):429-441; Hauer-Jensen et al., 2004, Crit Care Med, 32(5 Suppl):S325-S330). Due to endothelial cell heterogeneity, neutrophil recruitment to discrete organs may be regulated differently (Rossaint and Zarbock, 2013, Journal of Innate Immunity, 5(4):348-357). Whether PKCδ plays a similar role in neutrophil recruitment to different organs following radiation exposure is not known. PKCδ activation and PKCδ Tyr155 and Tyr 311 phosphorylation are critical in initiating endothelium activation and neutrophil recruitment following irradiation. Further, PKCδ nuclear translocation initiates apoptosis resulting in increased vascular permeability and tissue damage. The experiments described demonstrate that PKCδ inhibition decreases neutrophil recruitment, is organ protective, and decreases mortality. The effects of PKCδ inhibition on radiation-induced adhesion molecule expression and neutrophil influx in various organs are examined. Organ damage is assessed by tissue architecture (H & E staining), apoptosis (TUNEL and caspase 3 cleavage), and serum markers of organ damage.

The effects of irradiation are examined in the 6 animal group: 1) WT mice, 2) PKCδ-/- mice, 3) PKCδY155F KI mice, 4) PKCδY311F KI mice, 5) WT+SC administration of PKCδ TAT inhibitor (0.2 mg/kg) and 5) WT+SC PBS vehicle (6 mice/group). At 48 hours, 1 week, and 1 month post-irradiation, mice are euthanized and mesentery, lung and brain excised, and tissue sections prepared as described previously (Kilpatrick et al., 2011, Journal of Leukocyte Biology, 89(1):3-10; Mondrinos et al., 2014, The American Journal of Pathology, 184(1):200-13). Organs are formalin fixed, embedded in paraffin and sectioned at 5 um, coded and blinded sections are evaluated for histology (H &E staining), neutrophil infiltration (myeloperoxidase staining), and adhesion molecule expression as described previously (Kilpatrick et al., 2011, Journal of Leukocyte Biology, 89(1):3-10; Mondrinos et al., 2014, The American Journal of Pathology, 184(1):200-13). Systemic neutrophil counts are determined to ascertain if changes in tissue neutrophils are a reflection of decreased number of systemic neutrophils as a result of mouse phenotype (Gorovoy et al., 2009, Circulation Research, 105(6):549-556). ICAM-1 and VCAM-1 expression in tissue is assessed by immunofluorescence (Mondrinos et al., 2014, The American Journal of Pathology, 184(1):200-13). Apoptosis is analyzed in tissue sections by TUNEL assay (Roche Applied Science) and cleaved caspase 3 expression by immunostaining (Cell Signaling, Beverly, Mass.). Organ function is monitored by determining serum levels of LDH, alanine amino-transferase, alkaline phosphatase, aspartate aminotransferase, cardiac troponin I, BUN, and creatinine (Charles River Clinical Pathology Lab) and blood gases (i.e. PaO2, PaCO2, pH). For survival studies, the therapeutic effect of the PKCδ-TAT inhibitor is tested when the inhibitor is administered 24 hours post-IR as compared to vehicle. Mice are monitored for up to one month (18 mice/group). Kaplan-Meier survival curves are constructed with statistical analysis by the log-rank test.

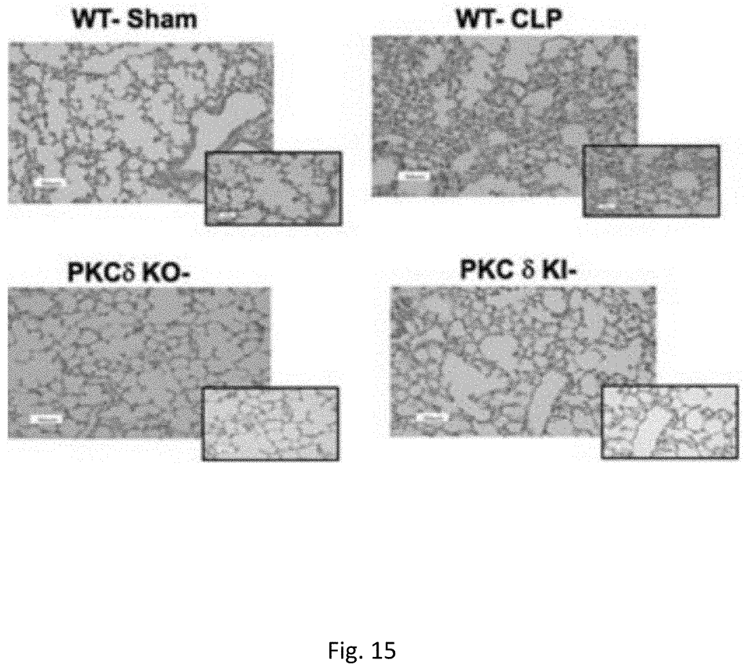

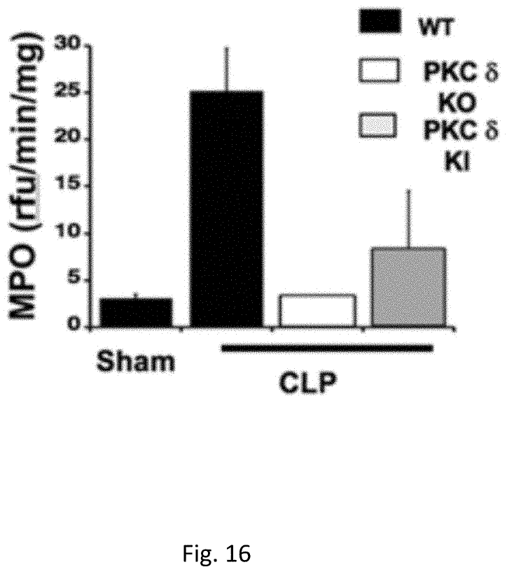

Decreased neutrophil-endothelial cell interaction as well as decreased permeability and endothelial cell damage in PKCδ-/- KO, PKCδY155F KI and PKCδY311F KI irradiated mice compared to WT is consistent with the data presented in FIG. 3 through FIG. 6. Neutrophil migration is organ specific and regulated by different mechanisms (Rossaint and Zarbock, 2013, Journal of Innate Immunity, 5(4):348-357; Kolaczkowska and Kubes, 2013, Nat Rev Immunol, 13(3):159-175). PKCδ inhibition decreased ICAM-1 and VCAM-1 expression and neutrophil migration (Kilpatrick et al., 2011, Journal of Leukocyte Biology, 89(1):3-10; Mondrinos et al., 2014, The American Journal of Pathology, 184(1):200-13). Decreased neutrophil extravasation in PKCδ-/- KO and PKCδY155F KI mice is consistent with decreased lung tissue damage and neutrophil migration in septic PKCδ-/- KO and PKCδY155F mice as compared to septic WT mice (FIG. 15 and FIG. 16). Decreased neutrophil infiltration and decreased apoptosis lead to decreased tissue damage and increased survival.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of treating or preventing radiation-induced injury comprising administering a PKCδ inhibitor to a subject in need thereof

2. The method of claim 1, wherein the PKCδ inhibitor is a PKCδ-TAT peptide inhibitor.

3. The method of claim 1, wherein the PKCδ inhibitor is administered to a subject undergoing a radiation-based therapy.

4. The method of claim 1, wherein the PKCδ inhibitor is administered to a subject exposed to high levels of radiation.

5. The method of claim 1, wherein the subject is human.

6. A method of decreasing neutrophil migration in a subject in need thereof, comprising administering a PKCδ inhibitor to the subject.

7. The method of claim 6, wherein the PKCδ inhibitor is a PKCδ-TAT peptide inhibitor.

8. The method of claim 6, wherein the PKCδ inhibitor is administered to a subject undergoing a radiation-based therapy.

9. The method of claim 6, wherein the PKCδ inhibitor is administered to a subject exposed to high levels of radiation.

10. The method of claim 6, wherein the subject is human.

11. Use of a PKCδ inhibitor in the manufacture of a medicament for the treatment or prevention of radiation-induced injury.

12. The use of claim 11, wherein the PKCδ inhibitor is a PKCδ-TAT peptide inhibitor.

13. The use of claim 11, wherein the radiation-induced injury is associated with a radiation-based therapy.

14. The use of claim 11, wherein the radiation-induced injury is associated with exposure to high levels of radiation.
